SEQUENCE LISTING (13 sequences)

SEQ ID NO: 1 - protein, 12 aa, Artificial
  Synthetic peptide derived from C2BL3
GGDYDKIGKNDA

SEQ ID NO: 2 - protein, 25 aa, Artificial
  Synthetic peptide derived from MARCKS
KKKKKRFSFKKSFKLSGFSFKKNKK

SEQ ID NO: 3 - protein, 10 aa, Artificial
  Synthetic peptide derived from Bradykinin
GRPPGFSPFR

SEQ ID NO: 4 - protein, 25 aa, Artificial
  Synthetic peptide derived from MARCKS
KKKKKRASAKKSAKLSGASAKKNKK

SEQ ID NO: 5 - protein, 25 aa, Artificial
  Synthetic peptide derived from MARCKS
AAAAAAFSFAASFALSGFSFAANAA

SEQ ID NO: 6 - protein, 10 aa, Artificial
  Synthetically derived fragment of homo sapiens C2BL3
DYDKIGKNDA

SEQ ID NO: 7 - protein, 14 aa, Artificial
  Synthetic precursor peptide derived from C2BL3
GGXDYDKIGKNDAX

SEQ ID NO: 8 - protein, 11 aa, Artificial
  Synthetic peptide derived from Bradykinin
XGRPPGFSPFR

SEQ ID NO: 9 - protein, 11 aa, Artificial
  Synthetic peptide derived from Bradykinin
KGRPPGFSPFR

SEQ ID NO: 10 - protein, 11 aa, Artificial
  Synthetic mutant peptide derived from Bradykinin
XGAPPGFSPFA

SEQ ID NO: 11 - protein, 11 aa, Artificial
  Synthetic peptide
XGGPPRFRPFS

SEQ ID NO: 12 - protein, 14 aa, Artificial
  Synthetic peptide derived from C2BL3
GGXDYDKDGKIDAX

SEQ ID NO: 13 - protein, 14 aa, Artificial
  Synthetic peptide derived from C2BL3
GGXDYDKIGKNDAX

(19) United States
(12) Patent Application Publication
(10) Pub. No.: US 2015/0093333 A1
(43) Pub. Date: Apr. 2, 2015

(54) COMPOSITIONS, METHODS AND USES FOR PEPTIDES IN DIAGNOSIS, PROGRESSION AND TREATMENT OF CANCERS

(71) Applicant: THE REGENTS OF THE UNIVERSITY OF COLORADO, body corporate, Denver, CO (US)

(72) Inventors: Hang Yin, Boulder, CO (US); Jonel P. Saludes, Boulder, CO (US); Leslie Morton, Boulder, CO (US)

(21) Appl. No.: 14/241,852

(22) PCT Filed: Aug. 30, 2012
(86) PCT No.: PCT/US12/53225

§ 371 Date: Aug. 29, 2014

Related U.S. Application Data
(60) Provisional application No. 60/528,959, filed on Dec. 11, 2003.

(51) Int. Cl. G01N 33/574; G01N 33/58; G01N 33/92

(52) U.S. Cl. CPC G01N 33/574; G01N 33/92; G01N 33/582; G01N 2405/04
USPC 424/9.6; 435/7.8

(57) ABSTRACT

Embodiments herein report compositions, systems, methods, and uses for diagnosing and/or treating a condition in a subject. In certain embodiments, one or more peptides can be used as biomarker detectors for predicting onset or progression of disease. Some embodiments of the present invention report peptides capable of associating with MVs for predicting onset or progression of cancer in a subject. Other embodiments include methods of generating and or modifying peptides of use herein. Yet other embodiments herein report biomarker detectors capable of detecting agents associated with cancer progression, for example, metastasis.

### PRIORITY CLAIM

The instant application is a PCT application that claims the benefit of U.S. Provisional Application No. 61/528,959 filed Aug. 30, 2011. The provisional application is incorporated herein by reference in its entirety for all purposes.

### FIELD

Embodiments herein report compositions, systems, methods, and uses for diagnosing and/or treating a condition in a subject. In certain embodiments, one or more peptides can be used as biomarker detectors for predicting onset or progression of disease. Some embodiments of the present invention report peptides capable of associating with microvesicles (MVs) for predicting onset or progression of cancer in a subject. Other embodiments include methods of generating and or modifying peptides of use herein. Yet other embodiments herein report biomarker detectors capable of detecting physiological features of cells associated with cancer progression, for example, metastasis.

### BACKGROUND

Many methods exist for predicting onset and progression of disease such as cancer. Biomarkers are used to predict onset, progression and often guide treatment of disease. Non-invasive biomarkers are sought after for these purposes.

Metastasis occurs when malignant tumor cells spread from their original site to other areas of the body, e.g., lung cancer to the brain. Recent reports have suggested that metastasis may be an early occurrence in cancer progression and is not its consequence of progression. It is believed that virtually all cancer cells have the potential to spread by metastasis, but their invasiveness and propensity to metastasize is affected by a variety of factors that include cell type, degree of differentiation, location, and many other less understood causes. Metastatic cancer-related death accounts for the majority of these deaths.

### SUMMARY

Some embodiments herein report compositions, methods and uses for agents capable of detecting exosomes or vesicles (referred to as microvesicles (MV)) of use to predict onset or progression of disease. Productions of MVs have been linked to certain medical conditions such as cancer metastasis. MVs capable of detection herein range from about 10 to about 150 nm in diameter. Compositions and methods for detecting the presence and level MVs disclosed herein can be used for determining whether a subject has a metastatic cancer to aide in early intervention or reduction of progression of the cancer. In other embodiments, peptide compositions herein can be used to identify whether a subject undergoing tumor removal has remaining cancerous cells. In accordance with these embodiments, a composition including one or more peptides disclosed herein can be used in a pharmaceutically acceptable form as a tracking agent for a health professional before, during or after tumor surgery.

Other embodiments disclosed herein concern compositions and uses for myristoylated alanine-rich C kinase substrate (MARCKS) protein or peptides derivatives thereof. In certain embodiments, compositions can include the entire MARCKS protein or peptides derived from particular domains of the protein. In certain embodiments, peptides can be derived from the Effector domain of MARCKS. In accordance with these embodiments, a composition comprising MARCKS protein or peptide derivative thereof can be used to detect MVs or other molecules with curved membranes for purposes of diagnosing disease or disease progression (e.g., cancer). MARCKS, MARCKS-derived peptide compositions, pharmaceutically effective compositions, or salts thereof can be used to detect MVs in a sample or in a subject in order to assess presence or level of metastasis in a subject suffering from cancer. In other embodiments, these compositions can be used to detect remaining cancer cells before, during or after tumor removal.

Some compositions concern other molecules or agents that associate with MVs such as synthetic peptides (mimetics) peptides or aptamers capable of associating with MVs that mimic the activity of peptides disclosed herein. Some embodiments concern peptides derived from proteins involved in membrane trafficking. In accordance with these embodiments, a membrane trafficking protein can be Synaptotagmin I or domains thereof. In certain embodiments, peptides disclosed herein can be cyclic or linear depending on affinity to associate with vesicles from about 10 to about 150 nm in diameter.

Other embodiments concern using trimeric forms of the peptide, Bradykinin, to analyze a sample from a subject or the subject for cancer progression. In addition, these novel forms of Bradykinin can be used as dimmers, trimers, pentamers or other suitable compound for detection of MVs in a sample or subject. In some embodiments, trimeric forms of Bradykinin are capable of detecting MVs and distinguishing them from normal conditions. In certain embodiments, these trimeric molecules are capable of identifying metastatic conditions in a subject having cancer.

Compositions and methods disclosed herein can be used alone or in combination with other methods for identifying or confirming metastatic tumors in a subject.

Subjects contemplated herein include humans and non-human mammals such as a dog or other domesticated animal.

### DESCRIPTION OF ILLUSTRATIVE EMBODIMENTS

### Definitions

As disclosed herein “modulate” can mean an increase, a decrease, upregulation, downregulation, an induction, a change in encoded activity, a change in stability or the like.

As disclosed herein where moieties are specified by their conventional chemical formulae, written from left to right, they equally encompass the chemically identical moieties that would result from writing the structure from right to left, e.g., —CH₂O— is equivalent to —OCH₂—.

### BRIEF DESCRIPTION OF THE DRAWINGS

The following drawings form part of the present specification and are included to further demonstrate certain embodiments herein. The embodiments may be better understood by reference to one or more of these drawings alone or in combination with the detailed description of specific embodiments presented.

FIGS. 1A-1B represent a three dimensional structure of a protein of certain embodiments disclosed herein (A) and an exemplary synthesis method for generating certain peptides of the instant application (B).

FIGS. 2A-2D represent exemplary fluorescence assays to detect interaction of certain peptides and lipid vesicles of some embodiments disclosed herein.

FIGS. 3A-3B represent exemplary nanoparticle tracking analyses (NTA) of various lipid vesicles treated with certain peptides contemplated herein.

FIGS. 4A-4D represent exemplary fluorescence enhancement assays with certain peptides contemplated herein (e.g., 500 nM) and lipid vesicles (e.g., 500 μM) or (A and B) and fluorescence anisotropy titration of these peptides with lipid vesicles (C and D).

FIGS. 5A and 5B represent fluorescence enhancement of certain peptides disclosed herein and Annexin-V after incubation with isolated microvesicles (A) and nanoparticle tracking analysis (NTA) with isolated microvesicles treated with these peptides at various concentrations (B).

FIGS. 6A-6C represent staining of certain wild type and mutant C. elegans with peptides of certain embodiments disclosed herein (A and B) and Annexin V (C).

FIGS. 7A-7B represent a schematic of preparation of monomer (A) and trimer (B) of certain peptides of some embodiments disclosed herein.

FIGS. 8A-8D represent exemplary fluorescence enhancement assays to identify interaction of certain monomeric and trimeric peptides disclosed herein with lipid vesicles.

### DETAILED DESCRIPTION

In the following sections, various exemplary compositions and methods are described in order to detail various embodiments of the invention. It will be obvious to one skilled in the art that practicing the various embodiments does not require the employment of all or even some of the details outlined herein, but rather that sequences chosen, proteins selected, samples, concentrations, times and other details may be modified through routine experimentation. In some cases, well-known methods or components have not been included in the description.

In accordance with embodiments of the present invention, there may be employed conventional molecular biology, protein chemistry, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Cancer proteomics can be used for identifying protein biomarkers for cancer screening wherein their level of expression can be detected as indicators of malignancy and metastasis. One of the technical challenges with this approach is that abundant blood proteins for example, albumins and fibrinogens can mask other less abundant biomarker proteins limiting their ability to be detected and therefore potentially producing false negative results.

Exosomes and vesicles (microvesicles (MV)) secreted by cancerous cells have been demonstrated as direct indicators of metastasis. Two unique features that differentiate microvesicles from normal cells are their size and phospholipid components. It has been reported that oncogenic cells contain 7-8% increase in phosphatidylserine on the outer leaflet of the membrane, whereas, normal cells contain a negligible 0.5-1% amount of phosphatidylserine. These MVs play important roles in cell-cell communication and cancer progression. These membrane-enclosed sacs of about 10 to about 1000 nm in diameter are thought to play important (patho) physiological roles in biological processes, for example, transfer of protein and genetic information, release of cytokines, and degradation of extracellular matrix, angiogenesis, and cell-to-cell communication during cancer progression. These particles are detected in human blood at concentrations of about 5 to about 50 ng/mL and are also found in other fluids. In certain embodiments described herein agents are designed to probe for and identify whether MVs are being secreted by cancer cells in a subject. In certain embodiments, a peptide-based probe to monitor the increased secretion of cell-derived s as potential, novel, and minimally invasive biomarker of cancer metastasis are generated. In certain embodiments, agents can include peptides, aptamers or other small molecules to detect MVs production in a subject having cancer.

In certain compositions and methods, peptides are generated that detect MVs in a biological sample of a subject or directly in the subject. Samples contemplated herein include, but are not limited to, biological samples such as skin, tissue, blood, bone, saliva or other samples. Certain peptides are designed to bind to highly curved MVs of about 100 nm or less, for example, to distinguish from an average control cell. Some embodiments regarding peptides can include linear or cyclic and/or modified peptides capable of easy tracking and identification. There can be certain advantages of using peptides as probes as they are readily modifiable to render natural and non-natural peptidomimetics with desired properties; and that are inexpensive to prepare even in large scale. In addition, the peptides of embodiments disclosed herein are trackable so are readily assessable when present in a sample or a subject.

In some embodiments, peptides can be used to detect MVs in a sample or in a subject having cancer. In other embodiments, cyclic peptides can include short cyclic peptides of about 8 to about 30-residue cyclic peptides. In certain embodiments, peptides of use as non-invasive biomarkers detector can be designed from molecules involved in cellular membrane trafficking. In accordance with these embodiments, peptides can be derived from Synaptotagmin I. Synaptotagmin I is a protein that is thought to mediate calcium-dependent regulation of membrane trafficking. In certain embodiments, peptides derived from Synaptotagmin I can be derived from domains of the protein. One domain is the C2B domain of Synaptotagmin I. In one embodiment, a 10-residue segment from Loop 3 of the cytoplasmic C2B domain of the transmembrane protein Synaptotagmin I (Syt1; pdb:IUOW) was used because C2B domain loops can bind to the membranes surfaces with defined curvature. In accordance with these embodiments, a peptide of 12 residues was identified (C2BL3C-HY, from the C2B domain, GGDYDKIGKNDA (SEQ ID NO:1)).

Another embodiment concerns an agent including the full-length myristoylated alanine-rich C kinase substrate (MARCKS) protein and peptide derivative thereof. The MARCKS peptide operates as a membrane curvature sensor by recognizing membrane bilayers in an extended conformation, which is driven by electrostatic interaction with negatively charged lipids. The MARCKS peptide can selectively target vesicles sized approximately around 100 nm, while recognizing the negatively-charged lipid component, overcoming the hurdles imposed to the current diagnostic technology of target selectivity. The MARCKS protein is about 87 kDa and therefore it is contemplated that smaller peptide may be more useful. In certain embodiments herein, the MARCKS effector domain was targeted for development of peptides that are capable of associating with MVs. Certain embodiments herein concern 10 to 40 AA length peptides derived from this domain. In one embodiment, a 25 residue Effector (KKKKKRFSFKKSFKLSGFSFKKNKK (SEQ ID NO:2)), domain derived peptide was identified and used for detecting MVs in a sample. It is contemplated that any peptide derived from this domain can be used to predict metastasis of a cancer by correlating presence or level of MVs in a sample from a subject having cancer (e.g., brain or melanoma).

Peptides disclosed herein can be generated by any methods known in the art. Some embodiments for generating peptides include SPPS synthesis with or without cyclization with solid phase chemistry. Certain solid phase chemistry techniques include Click chemistry. Cyclic peptides disclosed herein can include generating cyclic peptides about 10 to about 20 amino acids long. In certain embodiments, techniques known in the art to generate cyclic peptides can be used to generate a 12 residue cyclic peptide, the largest cyclic peptide reported to date using this technique. Certain embodiments include generating cyclic peptides from linear peptides immobilized on a solid substrate. Peptides generated by methods disclosed herein can be evaluated by spectroscopic techniques as well as being tested for binding to MVs by using liposomes to assess interaction with various sizes of molecules as well as animal models. In some embodiments, peptides and other MVs-associating molecules can be analyzed by Nanoparticle Tracking Analysis. Peptides disclosed herein are capable of detecting MVs (e.g., 10 nm to about 150 nm in diameter) in vitro or in vivo. In certain embodiments, cyclic peptides disclosed herein can be used to detect MVs production in a subject or in a sample from a subject in order to predict a condition or progression of a condition (e.g., metastasis). Samples from a subject can include solid or fluid samples. In some embodiments, the sample is a blood or plasma sample.

In certain embodiments, known protocols for peptide cyclization were used such as, lactam formation, lactonization, ring closing metathesis, disulfide bond formation, and “Click” chemistry. The key step in generating the 12-mer cyclic peptide disclosed herein was “Click” chemistry but modifications were performed to make the big ring because previous reports made 6-mer, 7-mer, 9-mer, and 11-mer (minor product) only. The following conditions were used to generate a larger quantity of the larger cyclic peptide: (1) low loading capacity resin of >0.2 mmol/g, (2) use of Copper (I) tetrakis(acetonitrile) hexafluorophosphate as the source of cuprous ion, (3) solvent composition of 93:7 NMP/H₂O that kept the sodium ascorbate in solution and ensured that the hydrophobic polystyrene solid support remained swollen.

In yet other embodiments, a trimeric peptide comprising peptides derived from Bradykinin; can be used to detect MVs in a sample. In certain embodiments, trimeric peptides of Bradykinin can be generated in large quantities and in a cost effective manner for use in identifying metastasis in a subject. Demonstrated herein are methods for using these compositions to analyze a sample or directly test a subject for progression of a cancerous condition.

### Cancer

Some embodiments disclosed herein concern predicting metastasis of a cancer in a subject. Cancers contemplated herein include liver, pancreas, cervical, kidney, corneal, lung, stomach, colon, breast, uterine, prostate, bone, skin (e.g., melanoma), and brain. In certain embodiments, MVs associated with these cancers can be screened for in order to diagnose metastatic state of the cancer. For example, embodiments herein can be used to study brain and skin cancer metastasis in a subject by correlating presence and or level of MVs in a sample from a subject having brain or skin cancer (e.g., melanoma).

### Metastasis

Metastasis is the spread of a cancer or a disease from one organ or part to another non-adjacent organ or part. It was previously thought that only malignant tumor cells and infections have the capacity to metastasize; however, this is being reconsidered due to new research. After the tumor cells come to rest at another site, they re-penetrate through the vessel or walls, continue to multiply, and eventually another clinically detectable tumor is formed. This new tumor is known as a metastatic (or secondary) tumor. Metastasis is one of three hallmarks of malignancy (contrast benign tumors). Most tumors and other neoplasms can metastasize, although in varying degrees (e.g., basal cell carcinoma rarely metastasizes).

When tumor cells metastasize, the new tumor is called a secondary tumor, and its cells are like those in the original tumor. For example, if breast cancer metastasizes to the lungs, the secondary tumor is made up of abnormal breast cells, not of abnormal lung cells. The tumor in the lung is then called metastatic breast cancer, not lung cancer.

### Routes of Metastasis

Main sites of metastases for some common cancer types. Primary cancers are denoted by “cancer” and their main metastasis sites are denoted by “metastases”.

Metastasis can occur by several routes such as spread into body cavities. This can occur by seeding surface of the peritoneal, pleural, pericardial or subarachnoid spaces. For example, ovarian tumors spread transperitoneally to the surface of the liver. Mesotheliomas can spread through the pleural cavity. Another way is by invasion of lymphatics. This invasion can be followed by the transport of tumor cells to regional nodes and ultimately to other parts of the body; it is common in initial spread of carcinomas. Another way is hematogenous spread. This mode is common to sarcomas but it is the favored route in certain carcinomas (e.g., those originating in kidneys). Because of their thinner walls veins are more frequently invaded than arteries and metastasis follows the pattern of the venous flows. Another way is by transplantation. This mode is by a mechanical transport of fragments of tumor cells by surgical instruments during operation or the use of needles during diagnostic procedures.

Cancer cells may spread to lymph nodes near the primary tumor. This is called nodal involvement, positive nodes, or regional disease. Localized spread to regional lymph nodes near the primary tumor is not normally thought of as metastasis, although this is a sign of worse prognosis. It is proposed that this spread may also involve the production of MVs. Transport through lymphatics is the most common pathway for the initial dissemination of carcinomas.

### Proteins and Peptides

### Protein Methodologies

Any method known in the art for identifying, isolating, modifying and assaying proteins or peptides herein are contemplated.

### Expressed Proteins

Some embodiments of the present invention concern generating proteins or peptides using recombinant technologies. Any method known in the art can be used for generating proteins or peptides disclosed herein. For example, certain proteins or peptides can be expressed in large quantities and purified. Examples of expression systems known to the skilled practitioner in the art include bacteria such as E. coli, yeast such as Pichia pastoris, baculovirus, and mammalian expression systems. A complete gene can be expressed or, alternatively, fragments of the gene encoding portions of polypeptide can be produced.

In certain embodiments of the invention, peptides and proteins disclosed herein can be used to detect MVs. Such peptides have features that associate with MVs. Genes or gene segments that encode polypeptides may be inserted into an expression vector by known subcloning techniques and expressed in a vector

Some proteins and peptides contemplated herein can be modified for tracking purposes. For example, tags may be added for tracing the peptides or tracking binding capability of the peptides to for example, microvesicles. In certain embodiments, fluorescent tags may be added to a peptide disclosed herein for inexpensive and rapid tracing of microvesicles

Certain embodiments herein describe using tracking and/or linking agents to follow peptides when associating with MVs. Potential tracking agents that can be linked to peptides can include, but are not limited to, one or more of fluorescent organic molecules such as Tryptophan, Coumarin, Fluoresceins (e.g., FITC), Nitrobenzoxadiazole, Alexa Fluor™ dyes (e.g., Alexa Fluor 647™) or others known in the art and Quantum Dots. Possible linking agents include, but are not limited to, Polyethylene glycols (PEGs) and similar polymeric compounds, or Alkyl chains (e.g., Aminohexanoic acid and similar compounds), and short peptides (e.g., GG) or other known linking agents.

Other embodiments concern generating polypeptides using synthetic methods. Such peptides are usually at least about six amino acid residues in length. Any method known in the art can be used to generate synthetic peptides. Amino acid sequence variants of the polypeptide may also be prepared. These may, for instance, be minor sequence variants of the polypeptide which arise due to natural variation within the population or they may be homologues found in other species. Sequence variants may be prepared by standard methods of site-directed mutagenesis or other method.

Amino acid sequence variants of a polypeptide contemplated herein may be substitutional, insertional or deletion variants. Deletion variants lack one or more residues of the native protein which may not be critical for function. Substitutional variants typically contain an alternative amino acid at one or more sites within the protein, and may be designed to modulate one or more properties of the polypeptide such as stability against proteolytic cleavage. Substitutions preferably are conservative, that is, one amino acid is replaced with one of similar size and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

It is contemplated herein that peptides can be generated based on their ability to associate with microvesicles by for example, binding to smaller diameter vesicles (e.g., 10 nm to 150 nm diameter scale or 30 nm to 100 nm scale) by detecting curvature of the vesicle versus that of a larger vesicle of a few to several hundred nanometer.

Another method for the preparation of the polypeptides according to embodiments disclosed herein is the use of peptide mimetics. Mimetics are peptide-containing molecules which mimic elements of protein or peptide secondary structure. The underlying rationale behind the use of peptide mimetics is that the peptide backbone of proteins exists chiefly to orient amino acid side chains in such a way as to facilitate molecular interactions. A peptide mimetic is expected to permit molecular interactions similar to the natural molecule.

The engineering of DNA segment(s) for expression in a prokaryotic or eukaryotic system may be performed by techniques generally known to those of skill in recombinant expression. It is believed that virtually any expression system may be employed for these techniques.

### Site-Specific Mutagenesis

Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Any method known in the art related to site-specific mutagenesis is contemplated of use herein.

### Protein Purification

Further embodiments concern purification, and in particular embodiments, the substantial purification, of a protein or peptide. The term “purified protein or peptide” as used herein, is intended to refer to a composition, isolatable from other components, wherein the protein or peptide is purified to any degree relative to its naturally-obtainable state, e.g., relative to its purity within a cell extract. A purified protein or peptide therefore also refers to a protein or peptide, free from the environment in which it may naturally occur.

Generally, “purified” will refer to a protein or peptide composition which has been subjected to fractionation to remove various other components, and which composition substantially retains its expressed biological activity. Where the term “substantially purified” is used, this will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. Actual units used to represent activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in the most purified state. It is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater-fold purification than the same technique utilizing a low pressure chromatography system. Methods exhibiting a lower degree of relative purification may have advantages in total recovery of protein product, or in maintaining the activity of an expressed protein.

It is known that the migration of a polypeptide may vary, sometimes significantly, with different conditions of SDS/PAGE. It will therefore be appreciated that under differing electrophoresis conditions, the apparent molecular weights of purified or partially purified expression products may vary.

### Aptamers

In certain embodiments, molecules of the present invention may be detected by binding agent of use may be an aptamer. Methods of constructing and determining the binding characteristics of aptamers are well known in the art. For example, such techniques are described in U.S. Pat. Nos. 5,582,981, 5,595,877 and 5,637,459, each incorporated herein by reference.

Aptamers may be prepared by any known method, including synthetic, recombinant, and purification methods, and may be used alone or in combination with other ligands specific for the same target. In general, a minimum of approximately 3 nucleotides, preferably at least 5 nucleotides, are necessary to effect specific binding. Aptamers of sequences shorter than 10 bases may be feasible, although aptamers of 10, 20, 30 or 40 nucleotides may be preferred.

Aptamers need to contain the sequence that confers binding specificity, but may be extended with flanking regions and otherwise derivatized. In a further embodiment, the flanking sequence may comprise a specific sequence that preferentially recognizes or binds a moiety to enhance the immobilization of the aptamer to a substrate.

The aptamers used as starting materials in the process of the invention to determine specific binding sequences may be single-stranded or double-stranded DNA or RNA. In a preferred embodiment, the sequences are single-stranded DNA, which is less susceptible to nuclease degradation than RNA. In preferred embodiments, the starting aptamer will contain a randomized sequence portion, generally including from about 10 to 400 nucleotides, more preferably 20 to 100 nucleotides. The randomized sequence is flanked by primer sequences that permit the amplification of aptamers found to bind to the target. For synthesis of the randomized regions, mixtures of nucleotides at the positions where randomization is desired may be added during synthesis.

### Pharmaceutical Compositions

Embodiments herein provide for administration of compositions to subjects in a biologically compatible form suitable for pharmaceutical administration in vivo. By “biologically compatible form suitable for administration in vivo” is meant a form of the active agent (e.g., pharmaceutical chemical, protein, gene, antibody etc of the embodiments) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically active amount of a compound may vary according to factors such as the disease state, age, sex, and weight of the individual, and the ability of antibody to elicit a desired response in the individual. Dosage regima may be adjusted to provide the optimum therapeutic response.

Pharmaceutical compositions containing a protein or peptide fragment thereof, or analog thereof, or mutant thereof, or a functional derivative thereof (e.g., pharmaceutical chemical, protein, peptide of some of the embodiments) may be administered to a subject, for example by subcutaneous, intravenous, intracardiac, intracoronary, intramuscular, by oral administration, by inhalation, transdermal application, intravaginal application, topical application, intranasal or rectal administration. Depending on the route of administration, the active compound may be coated in a material to protect the compound from the degradation by enzymes, acids and other natural conditions that may inactivate the compound. In a preferred embodiment, the compound may be orally administered. In another preferred embodiment, the compound may be administered intravenously. In one particular embodiment, the composition may be administered intranasally, such as inhalation.

A compound (e.g., a peptide, protein, a protein complex, a fusion protein or mixture thereof) may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. It may be necessary to coat the compound with, or co-administer the compound with, a material to prevent its inactivation. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use may be administered by means known in the art. For example, sterile aqueous solutions (where water soluble) or dispersions and sterile powders for the extemporaneous preparation of sterile injectable solutions or dispersion may be used.

Sterile injectable solutions can be prepared by incorporating active compound (e.g., a compound that reduces serine protease activity) in the required amount in an appropriate solvent with one or a combination of ingredients enumerated above, as required, followed by filtered sterilization.

Aqueous compositions can include an effective amount of a therapeutic compound, peptide, epitopic core region, stimulator, inhibitor, and the like, dissolved or dispersed in a pharmaceutically acceptable carrier or aqueous medium. Compounds and biological materials disclosed herein can be purified by means known in the art. Solutions of the active compounds as free-base or pharmacologically acceptable salts can be prepared in water suitably mixed with a surfactant, such as hydroxypropylcellulose.

Upon formulation, solutions will be administered in a manner compatible with the dosage formulation and in such amount as is therapeutically effective. The formulations are easily administered in a variety of dosage forms, such as the type of injectable solutions described above. It is contemplated that slow release capsules, timed-release microparticles, and the like can also be employed. These particular aqueous solutions are especially suitable for intravenous, intramuscular, subcutaneous and intraperitoneal administration.

Active therapeutic agents may be formulated within a mixture to comprise about 0.0001 to 1.0 milligrams, or about 0.001 to 0.1 milligrams, or about 0.1 to 1.0 or even about 1 to 10 gram per dose. Single dose or multiple doses can also be administered on an appropriate schedule for a predetermined condition such as daily, bi-weekly, weekly, bi-monthly etc. Pharmaceutical compositions are administered in an amount, and with a frequency, that is effective to modulate side effects. The precise dosage and duration of treatment may be determined empirically using known testing protocols or by testing the compositions in model systems known in the art and extrapolating therefrom. Dosages may also vary with the severity of the condition. In certain embodiments, the composition range can be between 10 and 75 mg/kg introduced daily or weekly to a subject. A therapeutically effective amount of al-antitrypsin, peptides, or drugs that have similar activities as al-antitrypsin or peptides can be also measured in molar concentrations and can range between about 1 nM to about 2 mM.

Alternatively, in other embodiments, a sample such as a blood, saliva, tissue, urine or other sample can be obtained from a subject and examined for the presence, absence or level of MVs and compared to a control sample to assess whether a cancer in a subject is metastatic. In accordance with these embodiments, agents disclosed herein for such a diagnose can be further used to guide a surgeon to assure complete removal of a metastatic tumor and or identify MVs that can be pooled or filtered from the subject in order to treat the subject.

Kits are contemplated in certain embodiments disclosed herein for detecting the presence or level of MVs in a sample or in a subject having cancer. Some embodiments concern a kit having a composition comprising one or more peptides disclosed herein of use to detect metastasis in a subject. Other embodiments concern kits of use to assess completion of tumor/cancer cell removal of by a health professional before, during or after a surgical procedure.

### EXAMPLES

The following examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

### Example 1

### Generation of C2B Derived Peptides

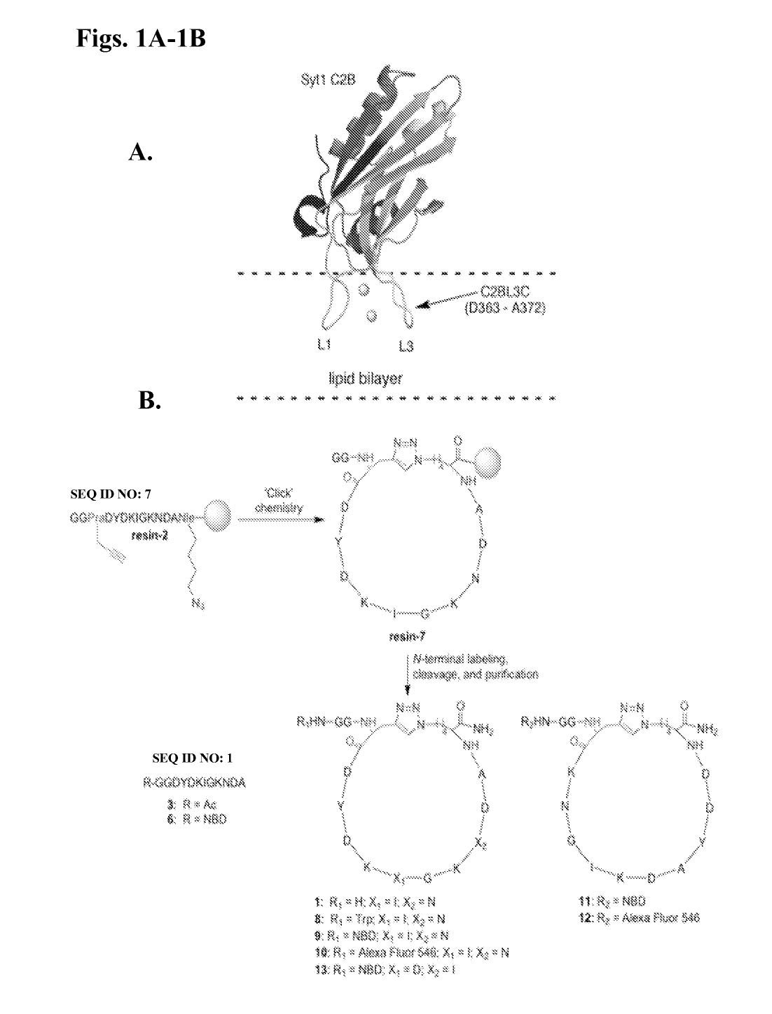

In one exemplary method, C2B peptides can be derived for uses described herein. As a component of the Soluble N-ethylmaleimide-sensitive factor Attachment protein Receptor (SNARE) complex, Syt1 is believed to mediate calcium-dependent regulation of membrane trafficking and fusion. Loops 1 and 3 of Syt1 C2B domain have been found to insert into membrane bilayers at a depth of 2.14±0.66 nm. The ability of membrane binding and insertion of Syt1 C2 domains is facilitated by a strong attraction with the negatively charged phospholipid head groups upon the formation of a Ca²⁺-complex that includes the highly conserved basic residue of loop 3 (Lys366 in pdb: luow). Furthermore, Syt1 prefers highly curved lipid vesicles (d=105 nm) to larger vesicles (d=252 nm) by sensing membrane curvature. Fluorescence quenching studies on C2B I367C mutant labelled at the C367 position demonstrated that C2B loop 3 penetrates the membrane bilayer, demonstrating its phospholipid binding ability. FIG. 1A illustrates the structure and conformation of Syt1 with loops 1 and 3 inserted into the lipid bilayer. Binding of Ca²⁺ represented by spheres and loops 1 and 3 allows Syt1 to adopt an active conformation to bind to membranes and sense curvature. Based on these observations, a 10-residue membrane-penetrating loop 3 (363-372) of the cytoplasmic C2B domain of the transmembrane protein Synaptotagmin-I (Syt1; pdb: luow) was obtained.

One peptide identified was a 12-residue molecule, Ac-GGDYDKIGKNDA-NH₂, (SEQ ID NO:1) derived from amino acids 363-372 of Syt1, wherein a flexible-GG-dipeptide linker was appended to the N-terminus of the peptides as a handle for functionalization, e.g., to facilitate the attachment of fluorophores. For example, tryptophan was chosen as one of N-terminal flurophores due to its easy availability of MD simulation parameters as well as ease of peptide synthesis to yield C2B-Trp peptide 4 (or 4). During the multiple nanosecond simulations of the C2B-Trp peptide-planar bilayer system, it was observed that the peptide invariably moves from the water layer and adheres onto the surface of the membrane (data not shown), suggesting that the membrane bilayer provides a favorable binding surface for the peptide.

Peptides C2B-Ac 3 (or 3), C2B-Trp 4 (or 4), C2B-Cou 5 (or 5), and C2B-NBD 6 (or 6) were synthesized using for example a Liberty microwave-assisted solid phase peptide synthesizer following the standard Fmoc chemistry. Rink amide resin and all reagents were purchased from commercial sources and used as received. Typically, 0.1 mmol of Rink amide MBHA resin (loading capacity=0.67 mmol g⁻¹) and 5 equiv. each of the Fmoc-protected amino acids (0.5 mmol), HBTU (0.5 mmol), and DIPEA (0.5 mmol) in 4 mL DMF were used for the coupling step. Reaction conditions were as follows: power=25 W, hold time=5 min, temperature=75° C. Fmoc deprotection required 7 mL of 5% piperazine and 0.1 M HOBt in DMF. All coupling and deprotection processes were repeated to ensure complete loading and deprotection. N-terminal-modified peptides were prepared using Ac₂O, Fmoc-L-Tryptophan 7-Hydroxycoumarin-3-carboxylic acid, 4-Chloro-7-nitrobenzo-2-oxa-1,3-diazole (NBD) following the solid-phase coupling method described above or a previously reported technique to yield C2B-Ac 3 (or 3), C2B-Trp 4 (or 4), C2B-Cou 5 (or 5), and C2B-NBD 6 (or 6). The peptides were cleaved from solid support using 10 mL of 82.5/5/5/5/2.5 TFA/thioanisole/phenol/H₂O/ethanedithiol mixture for 3 h followed by precipitation with cold Et₂O. The crude peptides were purified by reverse phase HPLC (Zorbax RP-C8, 9.4×250 mm; 5-50% aqueous MeCN with 0.1% TFA) with detection set at 210 and 280 nm. Eluates were concentrated and lyophilized to yield solid TFA salt of the peptides. The purified peptides were characterized by MALDI-TOF-MS: C2B-Ac 3, [M+H]⁺ calculated for C₅₄H₈₅N₁₆O₂₁⁺, 1293.6; found, 1293.5; [M+Na]⁺ calculated for C₅₄H₈₄N₁₆NaO₂₁⁺, 1315.6; found, 1315.5; C2B-Trp 4, [M+H]⁺ calculated for C₆₃H₉₃N₁₈O₂₁⁺, 1437.7; found, 1438.1; [M+Na]⁺ calculated for C₆₃H₉₂N₁₈NaO₂₁⁺, 1459.7; found, 1460.1; C2B-Cou 5, [M+H]⁺ calculated for C₆₂H₈₇N₁₆O₂₄⁺, 1439.6; found, 1439.9; [M+Na]⁺ calculated for C₆₂H₈₆N₁₆NaO₂₄⁺, 1461.6; found, 1461.9; C2B-NBD, 6 [M+H]⁺ calculated for C₅₈H₈₄N₁₉O₂₃⁺, 1414.6; found, 1415.0; [M+Na]⁺ calculated for C₅₈H₈₃N₁₉NaO₂₃⁺, 1436.6; found, 1437.0. ¹H NMR spectra were also recorded using a 500 MHz spectrometer at 293 K in 9:1 H₂O/D₂O (referenced to HDO, δH=4.79) as additional characterization.

FIG. 1B illustrates schematic representation of the solid phase synthesis of cyclic peptides derived from amino acids 363-372 of Syt1. Peptide cyclization has been demonstrated to increase binding to intended targets. Cyclization of a peptide can be achieved through lactam formation, lactonization, ring closing metathesis, and disulfide bond formation. ‘Click’ chemistry has been shown to achieve orthogonal, site-specific cyclization between azide- and alkyne-functionalized residues. It has been also adopted to achieve peptide macrocyclization on solid support resulting in the preparation of i to i+5, i to i+6, i to i+8, and i to i+10 cyclic peptides.

One of the peptides illustrated in FIG. 1B is a 12-residue cyclic peptide C2BL3C represented by 1. It was designed by side chain-mediated head-to-tail cyclization by ‘Click’ chemistry. This peptide was synthesized from the resin-bound linear precursor, peptide 2 (or 2), GGPraDYDKIGKNDANle(ε-N₃) (C2BL3L, resin-2) (SEQ ID NO:7), where Pra is L-propargylglycine and Nle(ε-N₃) is ε-azido norleucine. Cyclization of resin-2 by ‘Click’ chemistry was done at positions i and i+11 to give the cyclic peptide triazole resin-7. Progress of the reaction was monitored by standard techniques to identify completion of the reaction. The following solid phase ‘Click’ chemistry conditions were deemed to be optimum: (1) low loading capacity resin of <0.2 mmol g⁻¹ that prevented potential cross-linking and cyclodimerization (20); (2) use of [(CH₃CN)₄Cu]PF₆ as the source of cuprous ion; and (3) 93:7 NMP/H₂O that kept the sodium ascorbate in solution and ensured that the polystyrene solid support remained swollen. Analyses and comparisons of reversed phase HPLC chromatograms and ¹H NMR and FT-IR spectra confirmed the successful synthesis of peptide 1 (or 1) (data not shown). Portions of resin-bound cyclic peptide 7 were taken and N-terminally labeled with Trp or 7-nitrobenzo-2-oxa-1,3-diazole (NBD) to produce C2BL3C-Trp 8 (or 8) and C2BL3C-NBD 9 (or 9). For comparison purpose, a NBD-labeled linear peptide corresponding to the native C2B loop 3 region and an NBD-labeled scrambled analogue of 9, called C2BL3C-S-NBD (6 and 11, respectively) were prepared. A mutant of peptide 9 as an additional control, C2BL3C-I8D N11I-NBD 13 was prepared as well, where the point mutations correspond to the residues found in canonical EF-hand calcium-binding loops.

Peptide resin-2 can be synthesized using Rink amide resin and microwave-assisted solid phase synthesizer following the standard Fmoc chemistry. To make peptide resin-7, linear peptide precursor resin-2 (0.05 mmol) was swelled in 2.8 mL NMP for 5 min. A solution of Cu(I) tetrakis(acetonitrile) hexafluorophosphate (18.6 mg, 0.05 mmol) and Tris[(1-benzyl-1H-1,2,3-triazol-4-yl)methyl]amine (TBTA, 26.5 mg, 0.05 mmol) in 0.5 mL NMP was added to the resin, followed by sodium ascorbate (20 mg, 0.1 mmol in 250 μL H₂O) and 2,6-lutidine (10 μL, 0.1 mmol). Portions of the resin-7 were taken and labeled at the N-terminus with Trp and 4-Chloro-7-nitrobenzo-2-oxa-1,3-diazole (NBD) to yield 8 and 9, respectively. Peptides 12 and 13 were prepared following the same solid phase synthesis, cyclization, and labeling as described above to yield C2BL3C-S-NBD (12) and C2BL3c-I8D N11I-NBD (13). Resin-bound peptide 7 and its scrambled analogue were labeled with Alexa Fluor 546™ to yield C2BL3C-AF (10) and C2BL3C-S-AF (12), respectively.

A linear peptide precursor, GG-Pra-DYDKIGKNDA-Nle(ε-N₃)—NH₂ (SEQ ID NO:7) was synthesized following the microwave-assisted solid phase synthesis procedure as described above. Then the mixture was kept under a N₂ environment and was continuously stirred at low speed for 48 h at room temperature. The progress of the reaction was monitored by withdrawing aliquots of the resin, followed by peptide cleavage using TFA/TIPS/H₂O (95/2.5/2.5) and precipitation by cold Et₂O. Infrared (IR) spectra were recorded using Avatar 360 FT-IR (Thermo Scientific) and the disappearance of the azide band at 2300 cm⁻¹ in the IR spectrum indicated the completion of the reaction, which took 48 hours. The resin was sequentially washed with the following to remove residual copper ions: DMF, MeCN, H₂O, 0.1 M EDTA, H₂O, MeOH, CH₂Cl₂, MeOH. The peptide was cleaved from solid support using the cleavage cocktail described above for 3 h followed by precipitation with cold Et₂O. Portions of the resin-bound peptide were taken and labeled with Trp and NBD to yield C2BL3C-Trp 8 and C2BL3C-NBD 9, respectively. The crude peptides were purified by reverse phase HPLC (RP-C18, 10×250 mm; 5-50% aqueous MeCN with 0.1% TFA) and the eluates were lyophilized to yield solid TFA salts. The purified peptides were characterized by MALDI-TOF-MS: C2BL3C 7, [M+H] calculated for C₆₃H₉₆N₂₁O₂₂⁺, 1500.7; found, 1500.6; [M+Na]⁺ calculated for C₆₃H₉₅N₂₁NaO₂₂⁺, 1522.7; found, 1522.6; C2BL3C-Trp 8, [M+H] calculated for C₇₄H₁₀₈N₂₃O₂₃⁺, 1686.8; found, 1686.8; [M+Na]⁺ calculated for C₇₄H₁₀₇N₂₃NaO₂₃⁺, 1708.8; found, 1708.7; C2BL3C-NBD 9, [M+H]⁺ calculated for C₆₉H₉₉N₂₄O₂₅⁺, 1663.7; found, 1663.6; [M+Na]⁺ calculated for C₆₉H₉₈N₂₄NaO₂₅⁺, 1685.7; found, 1685.6. ¹H NMR spectra were also recorded using a 500 MHz spectrometer at 293 K in 9:1 H₂O/D₂O (referenced to HDO, δ_{H}=4.79) as additional characterization. The scrambled analogue of 7 and the mutant 13 (I8D N11I) were prepared following the same solid phase and cyclization protocol as described above using the sequences GGPraDYDKIGKNDA-Nle(ε-N₃) (SEQ ID NO:7) and GGPraDYDKDGKIDA-Nle(ε-N₃)—NH₂ (SEQ ID NO:12) as linear precursors, a portion was taken, and labeled with NBD to yield C2BL3C-S-NBD 11 (or 11): [M+H]⁺ calculated for C₆₉H₉₉N₂₄O₂₅⁺, 1663.7; found, 1663.3; [M+Na]⁺ calculated for C₆₉H₉₈N₂₄NaO₂₅⁺, 1685.7; found, 1685.3; [M+K]⁺ calculated for C₆₉H₉₈KN₂₄O₂₅⁺, 1701.7; found, 1701.3 and C2BL3C-I8D N11I-NBD 13 (or 13): calculated for C₆₉H₉₈N₂₃O₂₆⁺, 1664.7; found, 1664.8; [M+H]⁺.

### Example 2

### Preparation of Lipid Vesicles

In certain examples, lipid vesicles were prepared using extrusion through membranes with pore sizes of 30, 80, and 400 nm to cover a wide range of curvatures. In one example, small unilamellar vesicles (SUVs) composed of 100:0, 95:5, 9:1, and 8:2 mixture (mol/mol) of palmitoyl oleoyl phosphatidyl choline (POPC) and palmitoyl oleoyl phosphatidyl serine (POPS) were prepared as previously described. A lipid film was deposited on a glass vial by blowing down to dryness with Ar or N₂ a CHCl₃ solution of the lipids (Avanti Polar Lipids), followed by the removal of residual organic solvent under vacuum for several hours. The lipid film was hydrated overnight at 4° C. in PBS pH 7.4, the lipid suspension of multilamellar vesicles were subjected to five freeze-thaw cycles (except for extrusion through 400 nm pores), and followed by extrusion through polycarbonate membranes with pore diameters of 30, 80, and 400 nm to produce the desired SUVs using LiposoFast LF-50™. Representative samples from each unilamellar vesicle were taken and diluted with PBS to a lipid concentration of 10-20 μM and their sizes analyzed using Dynapro dynamic light scattering instrument, which was calibrated using commercially available 50 nm polystyrene beads (Polysciences). This process yielded vesicles with average diameters of 55±6, 115±6, and 511±54 nm, respectively, the smallest representing vesicles with the highest curvature (not shown).

Fluorescence Enhancement Assay of C2B Derived Peptides with Lipid Vesicles

In other exemplary methods, fluorescence enhancement assays were performed on fluorophore-conjugates of C2B-Ac 3, C2B-Trp 4, C2B-Cou 5, and C2B-NBD 6, to investigate the lipid vesicle binding property of 3 and its potential to differentiate lipid vesicles of different sizes following a known method. An increase in the observed fluorescence intensity and a blue shifted λₑₘ maximum are directly correlated to a change in the environment surrounding the peptide, e.g., from the polar aqueous solvent to the hydrophobic lipid vesicle, and indicate a peptide-lipid interaction. The peptides were prepared by microwave-assisted SPPS, purified by reversed phase HPLC, and characterized by MALDI-TOF-MS. Lipid vesicles of different sizes ranging from 0.03 to 0.08 μm (hence different radii of curvature) composed of 8:2 palmitoyl oleoyl phosphatidylcholine (POPC) and palmitoyl oleoyl phosphatidylserine (POPS) as models of exosomes were prepared using established protocols. Peptide 6 did not bind or sense membrane curvature as evident from the lack of change in the fluorescence intensity in the presence and absence of lipid vesicles. Peptides 4 and 5, however, did show significant binding and curvature sensing property with the highest binding preference to the surface of highly curved radius (0.03 μm vesicles) (data not shown). Fluorophores by themselves did not bind to lipid vesicles, as evident from the negligible change in the fluorescence intensity in the absence and presence of liposomes (data not shown).

An established fluorescence enhancement assay was followed with slight modifications to investigate the lipid vesicle binding property of the fluorophore conjugates of 1, peptides 8 and 9, and their potential to differentiate lipid vesicles of different sizes. An increase in the observed fluorescence intensity and a blue shifted λₑₘ maximum are directly correlated to a change in the environment surrounding the peptide, i.e., from the polar aqueous solvent to the hydrophobic lipid vesicle, indicating a peptide-lipid interaction. Lipid vesicles composed of 8:2 palmitoyl oleoyl phosphatidylcholine (POPC) and palmitoyl oleoyl phosphatidylserine (POPS) were used to represent the approximate phospholipid composition of cellular membranes. These vesicles were prepared by extruding a suspension of multilamellar lipid vesicles in PBS buffer (pH=7.4) through polycarbonate membranes. The diameters of these lipid vesicles were measured using dynamic light scattering (DLS) that was calibrated with 50 nm polystyrene beads. Syt1 was used as a positive control based on its property to bind to highly curved lipid vesicles. Peptides 6, 11, and 13 were used as negative controls. An optimal concentration of 0.5 μM was selected for all tested peptides (data not shown).

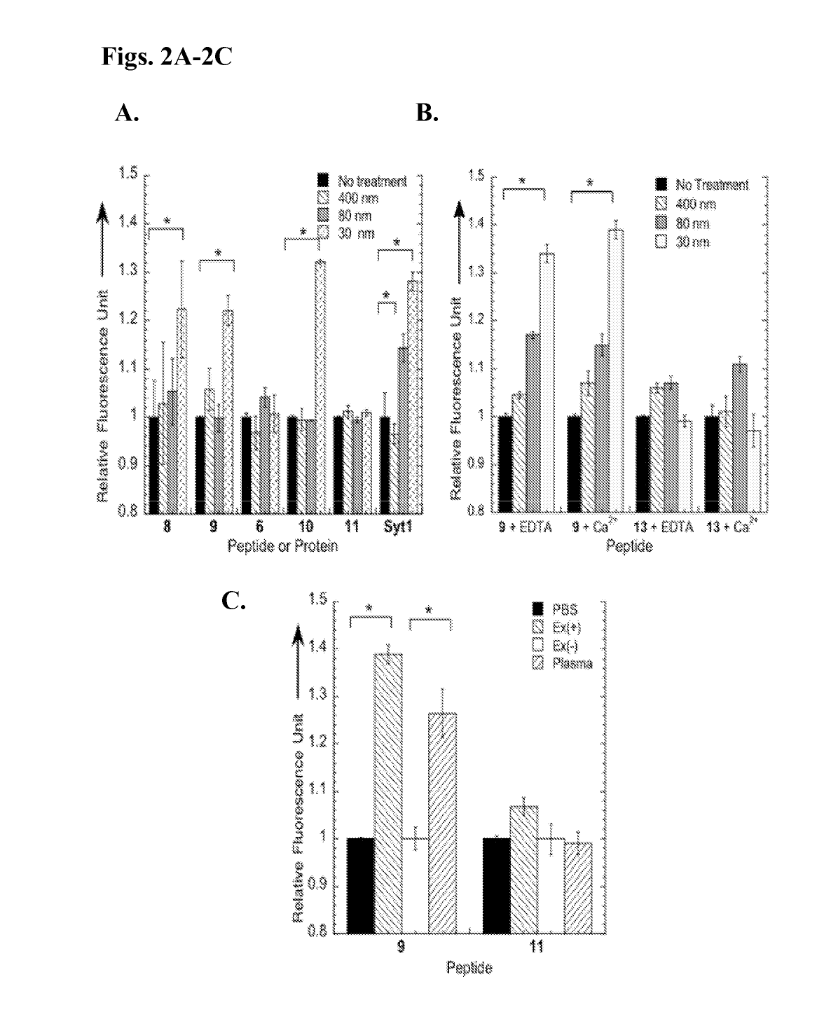

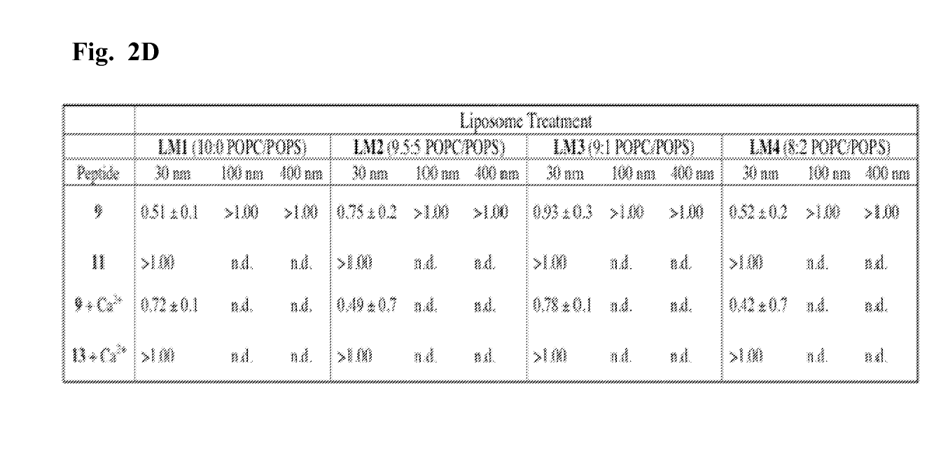

FIG. 2 illustrates exemplary fluorescence assays performed to assess the interaction of labeled-peptides on synthetic and biologically relevant lipid vesicles. FIG. 2A illustrates graphic data of peptide-liposome interactions of cyclic peptide-fluorophore conjugates and controls using 8:2 POPC/POPS vesicles extruded through 30, 80, and 400 nm pore membranes: C2BL3C-Trp 8, C2BL3C-NBD 9, linear C2B-NBD 6, C2BL3C-AF 10, C2BL3C-S-NBD 11, Synaptotagmin-I Syt1. Peptides 8, 9 and 10 bound to the lipid surface with the greatest curvature (d=55±6 nm) and with binding behaviors that were independent from the nature of the fluorescent tag. The linear peptide 6 and scrambled cyclic peptide 11 did not interact with lipid vesicles of any size (FIG. 2a), demonstrating that both cyclization and peptide sequence were essential for vesicle binding. The binding profiles to highly curved vesicles of 8, 9 and 10 are comparable, indicating that the fluorescence enhancement is not dependent on different fluorophores. This fluorescence enhancement was comparable to established curvature-sensing proteins: Syt1 (1.28±0.02 RFU with 30 nm vesicles, FIG. 2a) and the ALPS motif-bearing Golgi-microtubule-associated protein-210₁₋₃₈ and nuclear pore complex protein-133₂₄₅₋₂₆₇ (˜1.25 RFU with 29 nm liposomes). FIG. 2B provides graphic data of peptide-liposome interactions of C2BL3C-NBD represented by 9 and C2BL3C-I8D N11I-NBD represented by 13 with and without Ca²⁺ using 8:2 POPC/POPS vesicles extruded through 30, 80, and 400 nm pore membranes. 0.2 mM EDTA (ethylenediaminetetraacetate, disodium salt) and 1 mM CaCl₂ (Ca²⁺) was used respectively in the assay. Peptide 9, C2BL3C-NBD selectively binds to vesicles extruded through 30 nm pore membranes in the presence of EDTA or Ca²⁺. The lipid vesicle binding by C2BL3C did not need Ca²⁺, suggesting that rigidification by the covalent constraints rendered the cyclic peptide C2BL3C in an active conformation for curvature detection of phospholipid bilayers. Peptide 13, the I8D NM mutant of 9, did not bind to 8:2 POPC/POPS of any size in the presence of Ca²⁺.

In one correlation experiment, the above described findings were compared to biological specimens where the peptides were further tested on plasma samples from rats. These rats underwent inescapable tailshock stress as an acceptible model of exosome release (previously presented). Exosomes are nano-sized particles with diameters of ≦100 nm shed by various cell types, therefore providing a good biological model for testing the curvature-detecting property of the peptides.

### Isolation of Exosomes

Adult male Fisher 344 rats (Harlan, 8-9 weeks old) weighing approximately 250-275 g were used in all experiments. Animals were singly housed in clear Nalgene plastic cages (48×27×20 cm) and allowed access to food and water ad libitum. Temperature and humidity remained constant and animals were maintained on a 12-h:12-h light-dark cycle (lights on at 7:00 AM). Animals were allowed to acclimate to these housing conditions for 1 wk prior to any experimental manipulations and were handled each day. On the day of the experiment, the animals were exposed to 100, 1.5 mA, 5-second, intermittent, (average trial interval=60 s+/−25 s) inescapable tailshocks (stress). The animals were stressed as previously described. Immediately following stressor termination, animals were sacrificed. Trunk blood was collected in EDTA-coated tubes (13×75 mm) and plasma was isolated via 3000×g centrifugation at 4° C. for 15 min. The plasma was divided into 100 μL aliquots and warmed to 37° C. An equal volume of Thromboplastin-D pre-warmed to 37° C. was added to the plasma aliquots and incubated for 15 min. Fibrinogens were pelleted from the plasma following centrifugation at 10,000 rpm for 5 min at room temperature. The supernatant was removed and exosomes were precipitated out of the supernatant with the addition of 25 μL of ExoQuick™. Following an overnight incubation at 4° C., ExoQuick™ treated samples were centrifuged at 1,500×g for 30 min at 4° C. The supernatant was aspirated off and labeled as an exosome depleted fraction, called Ex(−). The remaining pellet that contained the exosomes was resuspended in 50 μL of phosphate buffered saline (PBS) and was called Ex(+).

### Identification of Exosomes

The blood plasma was fractionated to separate the exosomes from the plasma supernatant. The size range of exosomes is known and exosomes are characterized by the presence of a membrane-bound tetraspanin called CD63 and the membrane transport protein Rab5b. Particles expressing both proteins were captured by antibodies and quantified in a colorimetric endpoint assay. Individual wells in a ninety-six well plate were coated with 100 μL of 4 μg mL⁻¹ rabbit polyclonal Rab5B antibody (clone A-20, Santa Cruz Biotechnology, Santa Cruz, Calif.) in carbonate-bicarbonate buffer and incubated overnight at 4° C. Following three washes with phosphate buffered saline Tween (PBST), 100 μL of 2.5% bovine serum albumin (BSA) in PBS was added to each well and incubated overnight at 4° C. After the washes with PBST, 50 μL of Ex(+) or PBS was added to the wells and incubated overnight at 37° C. Following three washes with PBST, purified mouse anti-rat CD63 antibody was diluted to 8 μg mL⁻¹ in PBS and 100 μL was added to all the wells. After incubating at 37° C. for one h, the plate was washed three times with PBST and incubated at 37° C. with 100 μL of goat anti-mouse Poly-HRP (Pirece, Rockford, Ill.) diluted to 1:50,000 in 1% BSA in PBS. After three washes with PBST, the plate was developed with peroxidase detection for 10 min and the reaction was stopped with 1 M H₂SO₄. The optical densities (OD) were recorded at 450 nm using SpectraMax Plus384 microplate reader. Aliquots of Ex(+) and plasma were deposited on the surface of Formvar-carbon grids, air-dried, stained with Uranyl acetate, and the transmission electron microscopy images of isolated exosomes were recorded, which showed vesicles with d<150 nm with the majority of the particles having d<100 nm while the blood plasma showed a more heterogeneous mixture of a wide range of sizes from different kinds of particles (data not shown).

### Binding to Exosomes

In another example, the exosome-detecting ability of C2BL3C was tested by fluorescence enhancement compared to controls. FIG. 2C represents a histogram plot of assays performed to assess binding properties of C2BL3C-NBD 9 and C2BL3C-S-NBD 11 with exosomes and complete blood plasma. Ex(−) represents peptides treated with exosome depleted plasma supernatant, served as a negative control. Ex(+) represents peptides treated with isolated exosomes. PBS is another negative control used to treat peptides. Error bars represent SEM (n=3). * represents p<0.05 versus negative control. The Ex(+) treated peptide renders a higher fluorescence intensity at 1.39±0.02 RFU than the untreated peptide, which demonstrated that the synthetic phospholipid binding property of 9 was translated to the detection of exosomes. Peptide 9 also demonstrated the capacity to detect exosomes in blood plasma by showing a fluorescence intensity that was remarkably higher than the untreated 9 (1.27±0.05 RFU). In contrast, scrambled peptide 11 showed negligible binding. This demonstrates that the complex blood plasma matrix does not compromise the exosome-detecting ability of peptide 9. The peptide with the high affinity to exosomes shows the potential as a robust diagnostic tool for the detection of exosomes released in the peripheral blood of patients with metastatic cancer.

Fluorescence anisotropy was performed to measure the binding affinity of the peptides to liposomes. Peptides were titrated with liposomes of varying compositions of 10:0, 9.5:5, 9:1, 8:2 POPC/POPS, hereinafter called liposome model (LM) 1, 2, 3, and 4. The peptide partitions between the hydrophobic lipid bilayer and the aqueous solvent. Since the peptide unlikely forms a 1:1 complex with the lipid vesicle, the molar partition coefficient is often used. By definition, the K_{d}, apparent dissociation constant, is the reciprocal of the molar partition coefficient described as the lipid concentration where 50% of the peptide is bound. FIG. 2D is a table summarizing various results of peptides mixed with liposomes including the dissociation constant (K_{d}) values, in mM, of C2BL3C-NBD 9, C2BL3C-S-NBD 11, and C2BL3C I8D N11I-NBD 13 with liposomes of varying POPC/POPS compositions (mol/mol) extruded through 30, 100 and 400 nm pore membranes. Lower values indicate stronger binding. LM represents liposome model. 1 mM CaCl₂ was used to provide Ca²⁺. “n.d.” is used to represent “not determined”. The affinity of peptide 9 to lipid vesicles extruded through 30 nm pores was not affected by lipid composition, with K_{d} values of 0.51±0.1, 0.75±0.2, 0.93±0.3, and 0.55±0.2 mM for LM1, LM2, LM3, and LM4, respectively. Treatment with Ca²⁺ did not appear to significantly affect the affinity of 9 for 30 nm lipid vesicles (FIG. 2D), consistent with the findings in the fluorescence enhancement assay. By contrast, peptides 11 and 13 did not show significant affinity to 30 nm lipid vesicles of any lipid composition tested. Peptide 9 showed fairly weak binding (>1.00 mM) for 100 and 400 nm lipid vesicles. The observed binding of 9 for 30 nm vesicles is weaker relative to the property of Syt1 (K_{d}=0.151±0.6 and 0.263±0.18 mM for 105 and 252 nm vesicles, respectively). This is not surprising because peptide 9 was designed from only one of the components of the curvature sensing protein Syt1. Nonetheless, these findings showed that 9 had the ability to distinguish lipid vesicles of different curvatures regardless of the liposome composition. Lack of effective electrostatic interaction as a driving force in the peptide 9-liposome interaction, provided in the results presented in FIG. 2D, support that a driving force for the interaction is high curvature being a prerequisite for the observed size differentiation. Peptide 9 was able to distinguish lipid vesicle sizes which may be due to its ability to recognize lipid packing defects in highly curved lipid bilayers, a consequence of the mismatch between the phospholipid geometry and the curvature of the bilayer.

To verify that the origin of the differential peptide interaction towards lipid vesicles is due to the innate property of the peptide itself and not due to the fluorophores, ¹H NMR spectrometry investigations to probe the interaction of C2B derived peptides were carried out.

To probe the interaction of 3, 4, 5 and 6 with lipid vesicles, 0.03 μm vesicles were chosen for the NMR experiments because these showed the highest increase in fluorescence intensity upon interacting with 4 and 5. Visual inspection of the aromatic and amide region of the ¹H NMR spectra shows that 3 alone does not show changes in peak intensity or shape compared to untreated peptide indicating that the unlabeled peptide does not interact with the liposomes. This observation is in contrast to NMR profiles of peptides that are known to interact with liposomes. The spectra for 4 and 5 showed decrease in amide and aromatic ¹H resonance intensities in the presence of liposomes. Consistent to the fluorescence assay results above, 4 and 5 showed decrease in resonance intensity in the presence of liposomes compared to the untreated sample. These profiles indicate that 4 and 5 are interacting with the lipid vesicles and the corresponding decrease in peak intensities are indicative that these fluorophore-tagged peptides are binding to the macromolecule (liposomes). In contrast, the spectrum of liposome-treated 6 does not demonstrate the same profile as 4 and 5. Peptide 6 consistently failed to show any binding in both fluorescence and NMR experiments. The observation from the NMR experiments that the unlabeled peptide 3 does not interact with the lipid vesicles suggests that the observed differences in lipid binding arise specifically upon the inherent property of the labeled peptide, e.g., conjugation of fused aromatic, hydrophobic fluorophores at the N-terminus of 3 facilitated and enhanced the peptide binding to highly curved membrane.

To probe the interaction of 8 and 9 with lipid vesicles, vesicles with d=55±6 nm for the NMR experiments were chosen due to the fact that these vesicles showed the highest increase in fluorescence intensity upon interacting with peptides 8 and 9. Visual inspection of the aromatic and amide region of the ¹H NMR spectra showed that 1 treated with the lipid vesicles exhibited decreased peak intensity when compared to untreated 1 (data not shown), indicating that the unlabeled peptide also interacts with liposomes. By contrast, the spectra of liposome-treated, linear peptides 3 and 6 consistently demonstrated weaker binding in both fluorescence and NMR experiments. These demonstrate that both peptide sequence and cyclization, but not the fluorophores, are crucial for the observed curvature-detecting property of the cyclic peptide referred to as C2BL3C.

### Example 3

### Nanoparticle Tracking Analysis of C2B Derived Peptides

Nanoparticle tracking analysis (NTA) was previously used to observe individual lipid vesicle particles. NTA records videos in scatter (fluorescence independent) and fluorescence modes, the latter providing speciation to confirm the binding of fluorophore-labeled molecules on target particles. The NTA software analyzes the video and measures the size of each particle from direct observations of diffusion in a liquid medium, independent of particle refractive index or density. Although NTA can resolve and simultaneously measure a wide range of particle sizes at the same time, there is an inherent limitation of measuring a stochastic process in a finite sampling time (limited by time at which each particle is tracked), which may result to lesser peak resolution quality. The ability to observe and track nano-sized vesicles using the designed peptides can demonstrate the proof-of-concept of size selectivity.

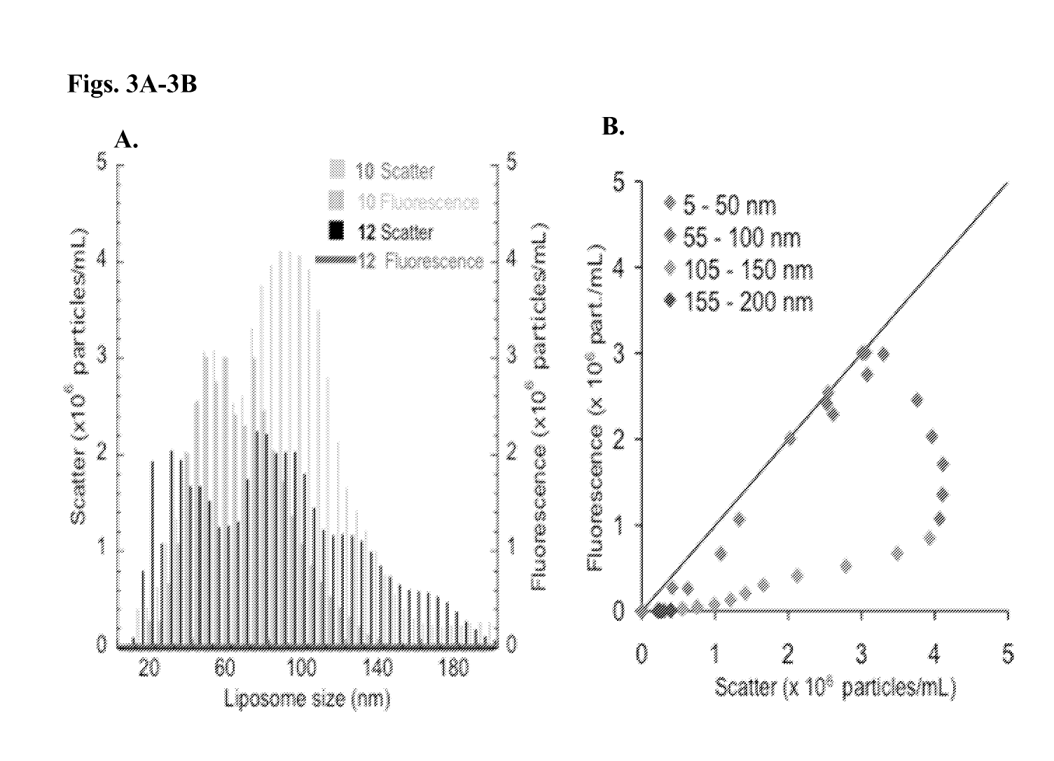

FIG. 3 represents data from an exemplary nanoparticle tracking analysis (NTA). C2BL3C and the cyclic scrambled peptide C2BL3C-S were labeled with Alexa Fluor 546 (required for NTA detection) to yield C2BL3C-AF 10 and C2BL3C-S-AF 12, respectively. Heterogeneous lipid vesicles composed of 8:2 POPC/POPS were prepared and treated with peptides 10 and 12. Videos were recorded under light scatter mode using 532 nm laser and then under fluorescence mode using a 560 nm filter to investigate if the particles were tagged by the peptides. FIG. 3A represents results of treating heterogeneous lipid vesicles composed of 8:2 POPC/POPS with peptide 10 or 12 in light scatter. The total vesicle count was 3.50×10⁸ mL⁻¹, with majority size ranging from 35-140 nm. Under fluorescence mode, the total particle count was 2.00×10⁸ mL⁻¹ and the vesicle sizes that were clearly tagged by peptide 10 were in the range of 30-95 nm (FIG. 3A). Compared to scatter mode, smaller particles (d<75 nm) were selectively labeled by peptide 10. FIG. 3B provides a parametric plot demonstrating the liposome size range that was preferentially labeled by peptide 10. Liposomes that scatter and fluoresce are represented by points that are on or close to the diagonal. The parametric plot (FIG. 3b) confirms that 10 preferentially tagged liposomes with a size range between 30 and 75 nm. In contrast, peptide 10 did not label large lipid vesicles, while peptide 12 did not tag any lipid vesicle size (FIG. 3A).

### Example 4

### Preparation of the MARCKS Derived Peptides

Based on previous reports, curvature-sensing proteins often have common features for sensing membrane curvature, including amphipathic alpha-helices (AHs), basic residues, and hydrophobic regions to insert into the lipid environment. MARCKS is an 87-kDa, intracellular protein that is present in cells at concentrations of 1-10 μM. It sequesters PIP₂ in the inner leaflet, regulating Phospholipase C signaling as well as binding to calcium-binding protein Calmodulin (CaM). The effector domain of the protein has been observed to interact with the membrane with an approximate μM level of affinity, increasing with higher molar concentration of acidic lipids, i.e phosphatidylserine. Upon binding to Calmodulin (CaM) in the presence of Ca²⁺, the binding of MARCKS to the membrane is reversible. It has also been reported that the membrane-binding affinity of MARCKS is driven by specific interaction with phosphatidylserine (PS), the membrane component carrying a negative charge while the protein secondary structure is not important. Based on these characteristics, truncated MARCKS peptide was thought to offer a starting point for sensing membrane curvature and lipid component in a vesicle. For example, MARCKS has (A) more than one of the common features found in curvature-sensing proteins are found in MARCKS, 13 basic residues and 5 hydrophobic residues, which have been shown to insert into the acyl chain region of the lipid environment; (B) the loss of the secondary and tertiary structure upon truncation is not critical to its membrane interaction, (C) the peptide region has high affinity to the PS-enriched membrane; and (D) the specificity of the peptide-lipid interactions can be verified using binding competition with CaM.

MARCKS peptide was synthesized using a CEM Liberty microwave-assisted peptide synthesizer following standard solid phase Fmoc chemistry. For fluorophore labeling, 4-chloro-7-nitrobenzo-2-oxa-1,3-diazole (NBD) or Alexa Fluor 546™ was conjugated to the N-terminus of the peptide via a flexible linker, E-aminohexanoic acid, using a previously reported coupling method. Kaiser test was performed to confirm the efficiency of the fluorophore labeling. At the end of the solid phase syntheses, the resin beads were washed with the following: dimethylformamide (DMF), dichloromethane (DCM), and methanol. The resin beads were then dried for one hour and the peptides cleaved using a water/trifluoroacetic acid (TFA)/triisopropylsilane (TIPS) cocktail (2.5/95/2.5) for 2-3 hours under inert conditions. The peptides were precipitated using chilled diethyl either. The peptides were then purified using reverse phase high performance liquid chromatography through a semi-prep C8 column. Following purification, peptides were lyophilized to produce a TFA salt powder. The prepared peptides were characterized by matrix-assisted laser-desorption ionization time-of-flight (MALDI) to confirm their identity.

Table 1 includes exemplary sequences of the synthetic peptides derived from the effector domain of myristoylated alanine-rich C-kinase substrate. To study the roles of particular amino acid residues, two mutant MARCKS peptides were prepared: (1) the five Phe residues that have been previously suggested to vertically insert into the bilayers were mutated to Ala to generate MARCKSmut1; and (2) the positively charged residues (Lys, Arg) were mutated to Ala to generate MARCKSmut2.

To observe the secondary structure of MARCKS-ED, circular dichroism (CD) spectroscopy analysis was performed for both the untreated and vesicle-treated samples. The peptide solutions were prepared at 10 μM in 10 mM phosphate buffer (pH=7.40) in the presence of 500 μM lipid vesicles (30 nm pore size with 20% PS). Circular dichroism spectra were recorded using the Chirascan CD spectrometer with a 1 mm path length quartz cuvette at 20° C. using phosphate buffer as a blank. The reading was then converted to molar residue ellipticity (θ). Five scans from 190 to 260 nm with data points taken every 1.0 nm were obtained and averaged for each sample. The results confirmed that MARCKS-ED adopts a predominantly random coil conformation (data not shown).

TABLE 1
Sequences of the MARCKS peptides

| | |
|---|---|
| MARCKS-ED: | KKKKKRFSFKKSFKLSGFSFKKNKK |
| | (SEQ ID NO: 2) |
| MARCKSmut1: | KKKKKRASAKKSAKLSGASAKKNKK |
| | (SEQ ID NO: 4) |
| MARCKSmut2: | AAAAAAFSFAASFALSGFSFAANAA |
| | (SEQ ID NO: 5) |

### Example 5

### Preparation of Synthetic Lipid Vesicles

In another exemplary method, lipid vesicles were employed to the curvature-sensing behavior of the MARCKS derived peptides with various sizes and lipid components. The membrane curvature is defined as the reciprocal of the diameter of a particular particle, e.g., smaller-sized vesicles present highly curved membrane bilayers. A series of lipid models were made to represent various lipid components that closely resemble the lipid composition of biological membranes. Small microvesicles have been observed to contain high concentrations of cholesterol and phosphatidylserine (PS) relative to normal cells. Dynamic light scattering (DLS) and negative-stain transmission electron microscopy (TEM) were performed to confirm the different vesicle sizes following the pressure-controlled extrusion process through different membrane diameter pores. Commercial polystyrene beads were used as the calibration standard for the vesicle size validation experiments. Furthermore, a time-course experiment was carried out to confirm the stability of the vesicles up to 16 hours. Three different membrane diameters were extruded for the following biophysical characterizations: 30 nm, 100 nm, 400 nm, reflecting different membrane curvatures.

### Interaction of Synthetic Liposomes and MARCKS-ED

In another exemplary method, a cosedimentation assay was performed to test the size differentiation behavior of MARCKS-ED. Two vesicle pore sizes, 100 and 400 nm, were used in this experiment because vesicles smaller than 100 nm were difficult to pellet even at high centrifugation speed. MARCKS-ED (10 μM) was incubated with 600 μM synthetic vesicles of sizes 100 nm and 400 nm. The positive control was the intact C2A-C2B cytoplasmic domains (C2AB) of rat Synaptotamin-1 (a.a. 96-421). C2AB (1.5 μM) was treated with CaCl₂ (1 mM) and incubated with 300 μM synthetic vesicles of sizes 100 nm and 400 nm. C2AB treated with vesicles was incubated at room temperature for 30 minutes, followed by centrifugation of 65,000 rpm for 45 minutes at 20° C. MARCKS-ED treated with vesicles was incubated at room temperature for 2 hours, followed by 75,000 rpm for 45 minutes at 20° C. The supernatant for each sample was collected as well as the pellets from the MARCKS-ED-treated samples and assayed on a pre-casted 12-15% Tris-Bis gel (Invitrogen, Eugene, Oreg.). The gel electrophoresis results of the supernatant and pellet samples collected after sedimentation with MARCKS-ED or C2AB, indicated that more MARCKS-ED or C2AB was pulled down by the 100 nm, PS-containing vesicles than by the larger 400 nm vesicles. Also, less MARCKS-ED was pulled down by the lipid vesicles without PS, suggesting that both the peptide-lipid binding and its curvature sensing depend on the presence of PS. Taken together, these results showed that, similarly to the C2AB protein, MARCKS-ED binds more tightly to highly curved vesicles containing PS.

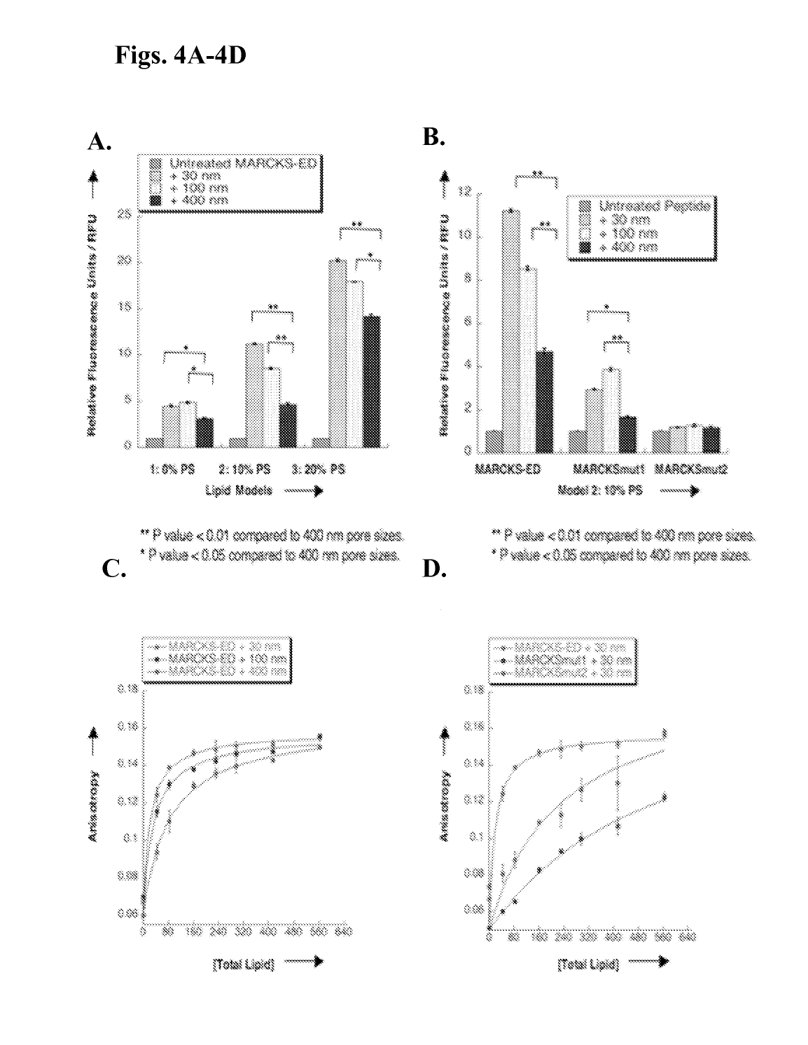

Next, a fluorescence enhancement assay was conducted to further quantify the curvature- and PS-sensing behavior of MARCKS-ED. Appropriate controls were performed to confirm that the fluorophore alone had no effect on the observed fluorescence enhancement (data not shown). FIG. 4 represents histogram data of fluorescence enhancement assay with and fluorescence anisotropy titration of the NBD labeled MARCKS derived peptides. Upon binding to lipid vesicles, the fluorescence intensity of NBD-MARCKS-ED increased due to vesicle binding and changes in the hydrophobic environment surrounding the fluorophore, concurring with a slight blue shift of the maximum emission wavelength. The emission spectra of all NBD-labeled peptides were recorded using a Fluorolog-3 fluorometer with λₑₓ=480 nm. The peptides and protein were tested at a concentration of 500 nM in PBS (pH 7.40) treated with 500 μM synthetic vesicles of different vesicles sizes. Fluorescence was observed with an emission range of 500-650 nm. The positive control C2AB (200 nM) from the rat Synaptotagmin-1 (Syn-1) protein (G374, residues 96-421) was treated with CaCl₂ (2.5 mM) and observed with λₑₓ of 275 nm and emission range of 300-450 nm. The untreated peptide and Ca²⁺-C2AB samples were corrected by the PBS (pH=7.40) blank solution for the peptides and PBS with Ca²⁺ for the Ca²⁺-C2AB sample. All samples were prepared and incubated overnight in 4° C.

FIG. 4A illustrates a histogram plot of fluorescence intensity of MARCKS-ED treated with lipid vesicles of various sizes. FIG. 4B illustrates a histogram plot demonstrating fluorescence intensity of different MARCKS derived peptides. As represented by the data, the fluorescence enhancement of MARCKS-ED treated with the 30 nm pore size lipid vesicles containing PS was significantly higher (statistic analysis was carried out using the ANOVA method). Samples treated with the 400 nm vesicles compared to samples treated with the 30 nm vesicles had a fluorescence intensity increase of approximately 1.5 fold, a change that is greater than the ones induced by the positive control protein, C2AB (FIG. 4A and data not shown). By contrast, MARCKSmut1 and MARCKSmut2 both demonstrated significantly reduced fluorescence enhancement and lacked the curvature sensing behavior observed in the wild type MARCKS-ED (FIG. 4B). Furthermore, the specificity of MARCKS-ED was confirmed by the observation that its binding could be partially reversed by the addition of CaM (data not shown). Last, the fluorescence intensity differences were less significant with vesicles containing no PS. Taken together, MARCKS-ED bound to highly curved vesicles containing PS, recognizing both shape and lipid composition simultaneously.

In another example, because fluorescence enhancement assays could not distinguish the contributions between different degrees of membrane penetration and binding affinity, fluorescence anisotropy assay was performed to specifically measure the binding affinity of the MARCKS derived peptides. The NBD-labeled peptides (1 μM) were titrated by synthetic liposomes of various sizes (30, 100, and 400 nm). Fluorescence anisotropy was recorded using a Fluorolog-3 fluorometer. The mixture was allowed to equilibrate for 2 minutes prior to the next titration. The excitation wavelength was set to λₑₓ=480 nm whereas the emission filter was set to λₑₘ=545 nm. The voltage was set to 250 V throughout the experiment. Blank PBS (pH=7.40) was titrated to NBD-labeled peptides as a negative control, where negligible anisotropy change was observed. The following equation was used to determine the dissociation constants as previously reported: F_{b}=Kₚ [L]/(1+K_{P}[L]), where F_{b} expresses the fraction of peptide bound to lipids, Kₚ expresses the molar partition coefficient and [L] expresses the lipid concentration.

Lipid vesicles were titrated to NBD-labeled MARCKS-ED, MARCKSmut1, or MARCKSmut2 peptides. Since the peptide partitions between the hydrophobic lipid bilayer and the aqueous solvent, the molar partition coefficient is often reported. By definition, the apparent dissociation constant (K_{d}), described as the lipid concentration where 50% of the peptide is bound, is the reciprocal of the molar partition coefficient (Kₚ). Fluorescence anisotropy assay results indicated efficient curvature sensing by MARCKS-ED. FIG. 4C represents an exemplary fluorescence anisotropy titration of MARCKS-ED with various lipid vesicles. FIG. 4D provides data of fluoresence anisotropy titration of various MARCKS derived peptides. Lipid vesicles used in the experiments represented by FIGS. 4C-4D contained 60% POPC:15% cholesterol: 15% POPE: 10% POPS. MARCKS-ED was found to bind to 30, 100, and 400 nm pore size lipid vesicles containing 10% PS with K_{d} values of 24±3, 42±13, 86±20 μM, respectively. As a comparison, C2AB illustrated a 1.9 fold increase in binding to smaller vesicles (105 nm) relative to the larger ones (252 nm). By contrast, MARCKSmut1 demonstrated greatly reduced binding affinity (FIG. 4D), confirming that the Phe residues were important. In addition, electrostatic interactions of MARCKSmut2 were also compromised. This data suggest that both aromatic and cationic residues are important for MARCKS-ED to sense highly curved vesicles, which is in agreement with what the interactions between MARCKS-ED and small, PS-enriched vesicles, that are driven by both electrostatic interactions and stabilization of lipid packing defects. Such defects would permit the Phe residue to insert and stabilize the highly curved membranes.

### Selectively Binding to PS-Enriched Microvesicles

In another exemplary method, having established the simultaneous curvature- and PS-sensing by MARCKS-ED in synthetic liposomes, MARCKS-ED was tested on detection of highly curved, PS-enriched particles in a complex biological system. Microvesicles (ø=0.03-1 μm) are highly curved lipid vesicles that shed into body fluids (e.g., blood, urine, ascitic fluid). These vesicles are released by cancerous cells in which lipid asymmetry is de-regulated, contributing to externalization and enrichment of PS on their outer leaflet. The microvesicle detecting ability of MARCKS-ED was investigated using plasma samples from a stressed rat model. Adult male Fisher 344 rats (Harlan, 8-9 weeks old) weighing approximately 250-275 grams were used in all experiments. Animals were allowed to acclimate to these housing conditions for 1 week prior to any experimental manipulations and were handled each day.

Microvesicles in these samples were characterized by TEM imaging, and ELISA of detection of the signature CD63 protein and the membrane transport protein Rab5b exposed on the surface. Particles expressing both proteins were captured by antibodies and quantified in a colorimetric endpoint assay.

These isolated microvesicles were measured by nanoparticle tracking analysis (NTA) to confirm their average size (ø=56 nm). Tracking analyses were performed using the NanoSight LM10-HS instrument (Amesbury, UK) equipped with a 532 nm laser and observed at scatter and fluorescence modes (filter=550 nm). The instrument was calibrated using commercially available 50 nm polystyrene beads. Parallel experiments were done on isolated exosomes and plasma that were not treated with MARCKS-ED conjugated with Alexa Fluor 546™ to confirm that the suspended particles do not autofluoresce. Alexa Fluor 546™-labeled mouse anti-rat CD63 and unconjugated Alexa Fluor 546™ were used as positive and negative controls, respectively. The profiles of MARCKS-ED-Alexa Fluor 546™ and Alexa Fluor 546™ in the absence of microvesicles were also investigated to confirm that these show negligible scatter and fluorescence background signals under identical NTA conditions.

Then, exposed PS was confirmed with an established PS-sensing protein, Annexin-V. Previous reports showed that upon binding to PS-enriched synthetic lipid vesicles, the fluorescence from W187 of Annexin-V increased. Monitored at λₑₓ=295 nm, Annexin-V (0.32 μM) was treated with CaCl₂ (3 mM) in TES buffer and with 500 μM lipid vesicles of varying PS content. An emission spectrum of 300-450 nm was recorded. Fluorescence enhancement was observed for the Annexin-V protein incubated with the isolated microvesicles, indicating protein-lipid interactions.

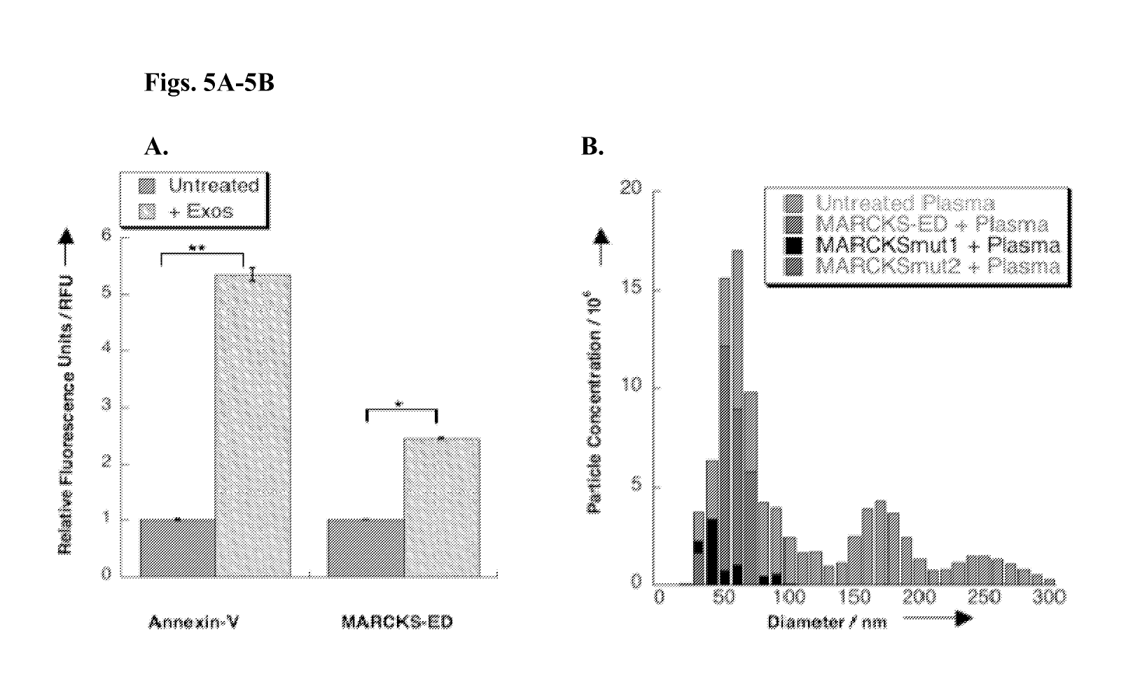

FIGS. 5A-5B represent histogram plots of exemplary fluorescence enhancement assay and NTA with MARCKS derived peptides.

FIG. 5A represents fluorescence enhancement assay with MARCKS-ED incubated with the isolated microvesicles. Peptide MARCKS-ED (500 mM) not treated with microvesicles was used as a negative control. Annexin-V (a positive control) and MARCKS-ED were incubated with the isolated rat microvesicles. Fluorescence was normalized to the negative control. MARCKS-ED was demonstrated to bind to these biological particles. The diameter size and particle count of the isolated microvesicles detected by MARCKS-ED was further quantified. Nanoparticle tracking analysis (NTA) uses a laser for scatter and fluorescence modes to track small particles by Brownian motion, providing a robust method for detecting nano-sized microvesicles. Under the fluorescence mode with the emission filter set for the Alexa Fluor 546™, the particles that bound to MARCKS-ED, MARCKSmut1, and MARCKSmut2peptides were observed.

FIG. 5B illustrates the results of NTA of microvesicles treated with MARCKS derived peptides. Fluorescently-labeled MARCKS-ED, MARCKSmut1 and MARCKSmut2 at concentrations of 55 nM were used to treat microvesicles in plasma from stressed rats. The untreated plasma samples were detected using the scatter mode and treated samples were monitored by tracking the fluorescence of Alexa Fluor 546™ conjugated to the MARCKS-ED MARCKSmut1 and MARCKSmut2 peptides. MARCKS-ED was found to selectively bind to small-sized microvesicles in whole plasma (FIG. 5B). MARCKSmut1 also showed some preferential binding to smaller vesicles but with much weaker fluorescence signal. MARCKSmut2 showed only negligible binding. Furthermore, blank samples were performed to rule out possible artifacts of background fluorescence from the peptides, the vesicles and the unconjugated dye. Taken together, these data demonstrate that MARCKS-ED can selectively detect biologically relevant micovesicles with highly curved, PS-enriched surfaces in the complex rat plasma.

### Example 6

### Staining PS-Exposing Membranes in C. elegans by MARCKS-ED

To demonstrate that PS-sensing plays a role in MARCKS-ED sensing to highly curved vesicles, MARCKS-ED was examined in a complex, animal system to selectively detect PS. Fluorescence staining assays were carried out in an acceptible C. elegans model with PS-exposing cell membranes. Inactivation of the tat-1 gene in C. elegans, which encodes for a phospholipid translocase that maintains PS plasma membrane asymmetry, resulted in PS externalization to the outer plasma membrane leaflet. NBD-MARCKS-ED was used to stain the dissected gonads of wild type (N2) animals, tat-1(tm3117) mutant animals, and engulfment-deficient ced-7 (n2094) mutant animals, respectively. 36 hour old hermaphrodite adult animals were collected and gently dissected by cutting their heads in a depression slide with a gonad dissection buffer (previously presented) to expose the gonads. The exposed gonads were then washed once in the dissection buffer and transferred to a dissection buffer containing 4 μM of Hoechst 33342 and 20 μM of NBD labeled MARCKS-ED peptide, 20 μM NBD-MARCKSmut1 peptide, or 10 nM Alexa Fluor 488-conjugated Annexin-V for 45 minutes. Gonads were washed one more time in the dissection buffer, placed on a 5% agarose pad, and visualized using a Nomarski microscope equipped with an epifluorescence detector.

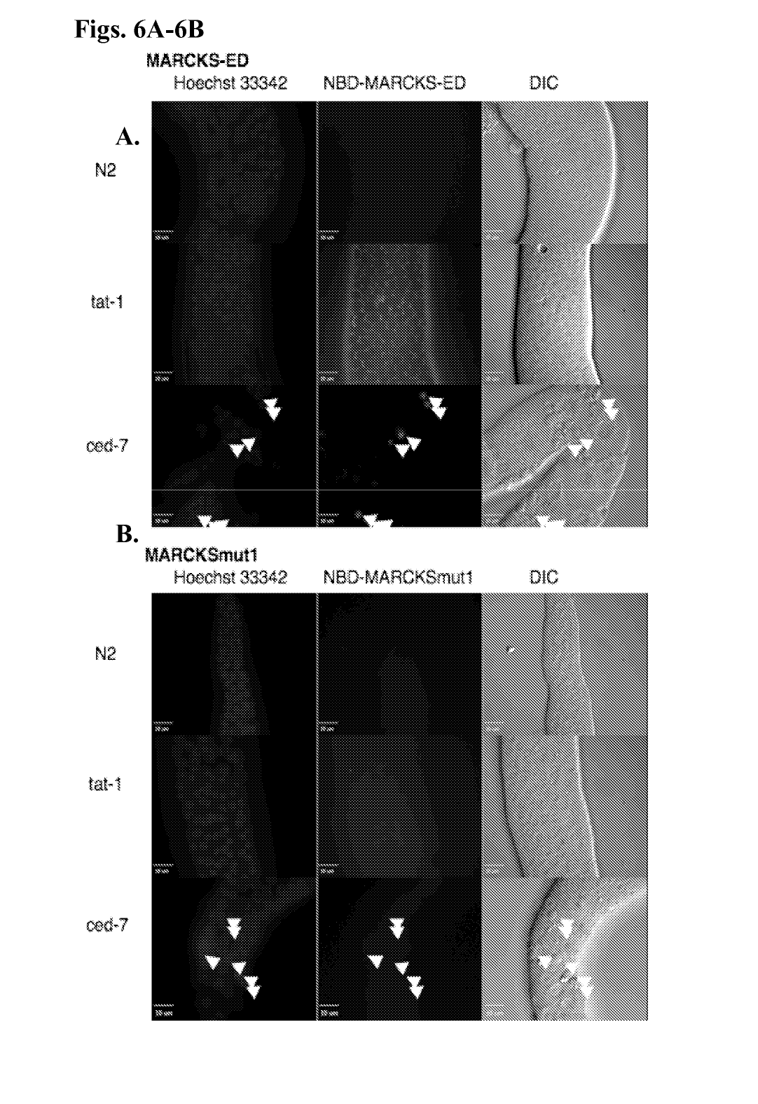

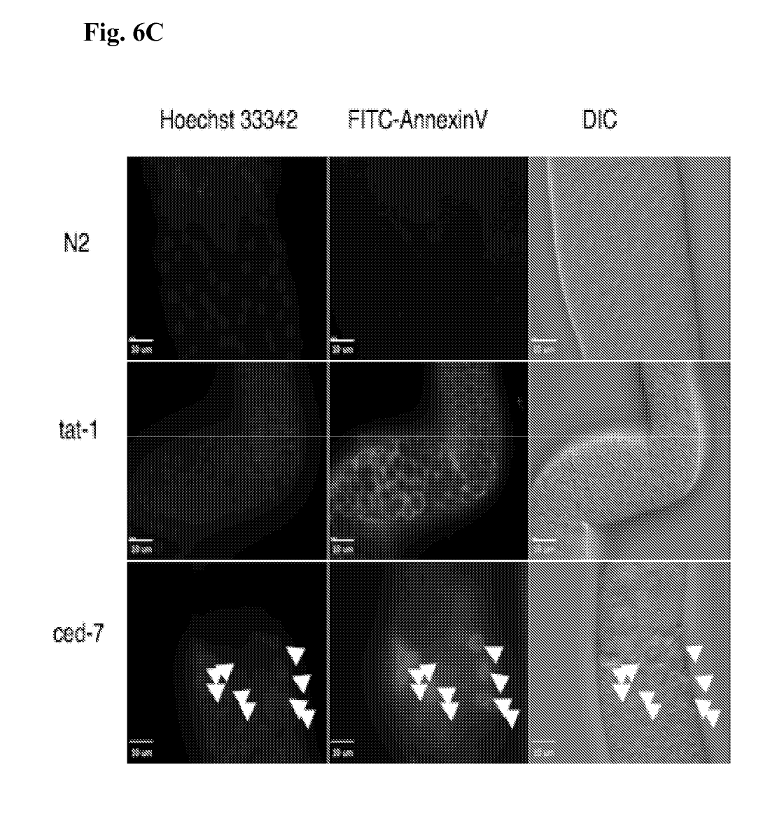

FIGS. 6A-6C illustrate exemplary staining results using MARCKS derived peptides and Annexin V. PS is typically kept in the inner leaflet of plasma membranes in living cells and is exposed on the cell surface only under certain cellular events, i.e., when cells undergo apoptosis or lose the ability to maintain the PS asymmetry with a tat-1 gene mutation. FIG. 6A represents the staining results with labeled MARCKS-ED in wild type and mutants C. elegans. NBD labeled MARCKS-ED recognizes PS exposed on the surface of all germ cells in the tat-1 (tm3117) mutant and un-engulfed apoptotic germ cell corpses in the ced-7(n2094) mutant. FIG. 6B represents the staining results with labeled MARCKSmut1 in wild type and mutants C. elegans. MARCKSmut1 did not appear to detect PS-exposed membranes in either tat-1 or ced-7 mutant animals, confirming that MARCKS-ED detects PS in a sequence-specific manner. In FIGS. 6A-6B, nuclei were stained with Hoechst 33342. Annexin-V, a known PS-sensor, was also used to stain the gonads of tat-1 and ced-7 mutant C. elegans. FIG. 6C demonstrates exemplary in-vivo fluorescence staining of C. elegans using Annexin-V. The exposed gonad of a wild type (top row of FIG. 6C) hermaphrodite C. elegans, a tat-1 (tm3117) (middle row) and a ced-7 (n2094) (bottom row) C. elegans were stained with FITC-AnnexinV and Hoechst33342. Hoechst33342 (4 μM), FITC-AnnexinV (10 nM) and Nomarski images are shown. Arrows in FIG. 6C indicate germ cell corpses stained with both Hoechst and FITC-AnnexinV protein. Scale bar=10 μm. The staining results with MARCKS-ED were comparable to the staining results observed with Annexin V, indicating that MARCKS-ED can be used as a new generation PS sensor to detect surface exposed PS in live animals.

MARCKS-ED was able to differentiate lipid vesicle sizes in both synthetic phospholipid models and microvesicles generated in the rat animal model. It was suggested that electrostatic interactions and aromatic Phe residues played a critical role in curvature sensing. MARCKS-ED recognized PS-enriched, curved membranes by filling in the defects in asymmetrically stretched bilayers with Phe, supported by the observation that its binding was greatly reduced in both MARCKSmut1 and MARCKSmut2 peptides. In vivo C. elegans studies confirmed specific detection of surface exposed PS by MARCKS-ED. These results shed insight to the poorly understood molecular mechanism of membrane curvature sensing. Moreover, MARCKS-ED could become a prototype for a new generation of peptide sensor that can simultaneously detect both PS and curvature to facilitate investigations of critical biological events.

### Example 7

### Preparation of Peptides Derived from Bradykinin

Bradykinin (BK) is a cationic peptide ligand for B1 and B2 G-protein coupled receptors, with amino acid composition of RPPGFSPFR (SEQ ID NO:6). BK was selected as the core molecule in this study because of the following reasons. It is believed that the conformation adopted by peptide ligands like BK is facilitated by interactions with membrane phospholipids prior to receptor binding and activation, suggesting that BK interacts with lipid bilayers. The general consensus suggests that BK exists as a random coil in aqueous solution but undergoes conformational modifications upon interaction with membrane lipids, which allows the accumulation of the active peptide conformation in the extracellular environment. Previous reports have demonstrated that BK penetrates membrane bilayers and manifests differential interactions with lipid vesicles and micelles, with a strong preference for mixtures with high anionic components. These findings suggest that an increase in the local peptide concentration at the vesicle surface is not only due to the cationic Arg at positions 1 and 9 but also due to the Phe at positions 5 and 8, supporting electrostatic- and hydrophobic-driven peptide-lipid interaction. These two properties are deemed to facilitate in differentiating phospholipid composition of vesicles. Proline residues induce a β-turn conformation that orients the Arg residues on the same face of the peptide to a claw-like shape. This conformation is believed to be critical for lipid recognition and attachment.

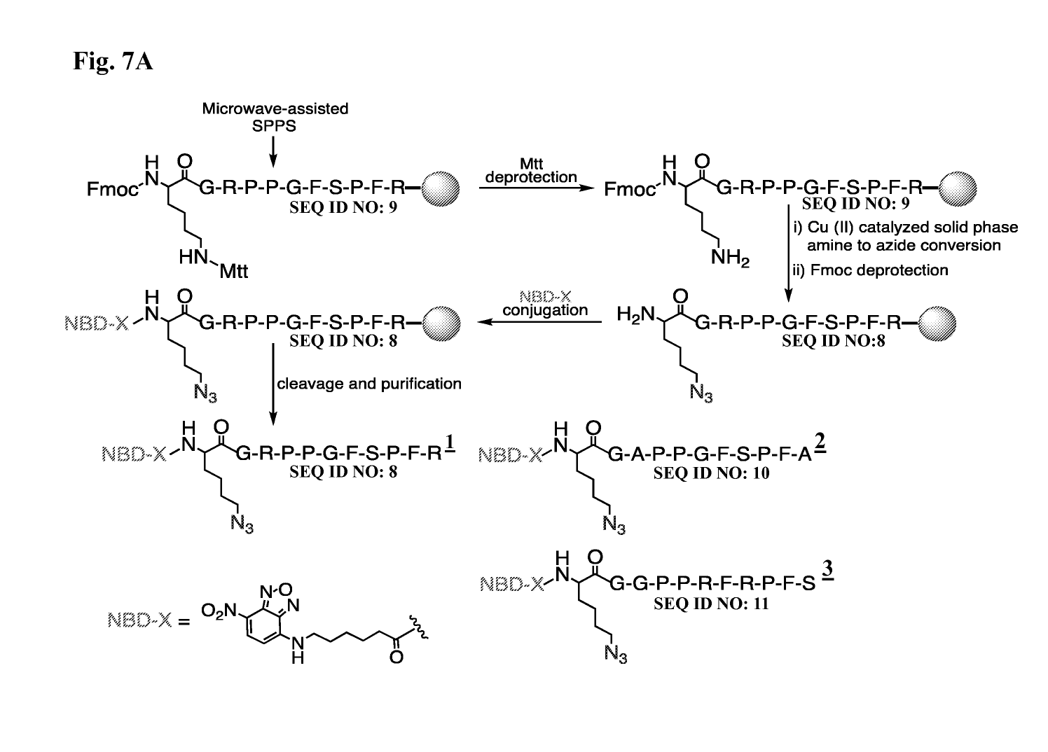

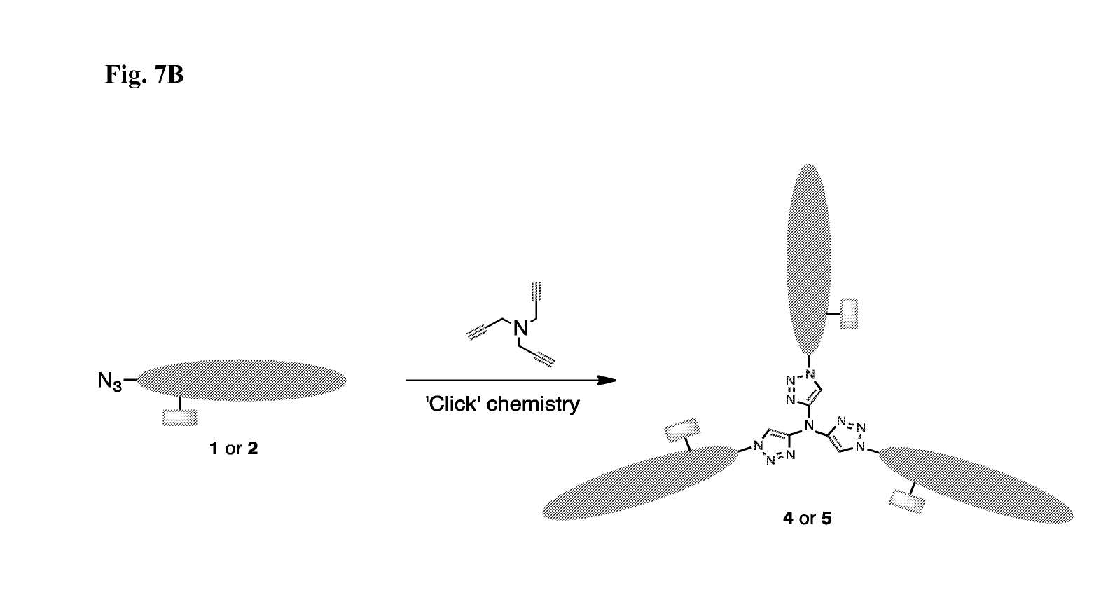

FIG. 7 is a schematic representation demonstrating a general process for the preparation of monomeric BK derivatives on solid support and solution phase trimerization by CuAAC.

Design of peptide 1 (or 1) was based on reports that N-terminal alteration on BK does not significantly affect its receptor binding activity, suggesting a conserved conformation. Gly was added as a spacer (GRPPGFSPFR (SEQ ID NO:3)), ε-azidolysine as orthogonal group for future functionalization, and 6-(N-(7-nitrobenz-2-oxa-1,3-diazol-4-yl) aminohexanoic acid (NBD-X) as fluorophore at the N-terminus. The 11-mer precursor of peptide 1 was synthesized using standard Fmoc chemistry and microwave-assisted solid phase peptide synthesis on Rink amide resin. The ε-amino group of the Lys residue was converted to azide by solid phase Cu(II)-catalyzed amine-to-azide conversion. Mtt protection was selectively removed using 94% CH₂Cl₂, 1% trifluoroacetic acid, and 5% triisopropylsilane (TIPS) (10 mL×3 min×10) at r.t., followed by thorough washing with CH₂Cl₂ and MeOH (5 mL×3). Following deprotection, the resin was swelled in a mixture of CH₂Cl₂/MeOH/H₂O/Et₃N, drained, and treated with CuSO₄ and freshly prepared TfN₃. The mixture was stirred for 24 h at r.t., followed by washing with MeCN, DMF, H₂O, 0.1 M EDTA, H₂O, MeOH, CH₂Cl₂, and MeOH. Kaiser test and FT-IR analysis were performed to confirm the presence of the azide moiety. The N-terminus was deprotected using 20% piperidine in 1-Methyl-2-pyrrolidinone (NMP) and conjugated with NBD-X. The peptide was cleaved from the resin using 85/5/5/5 TFA/H₂O/phenol/TIPS, purified by reverse phase HPLC, lyophilized, and characterized by mass spectrometry. In the same manner as described above, peptides 2 (RtoA mutant, or 2) and 3 (scrambled peptide, or 3) were prepared to investigate the effects of removing the positive charge and scrambling the position of Arg residues in the peptide. Peptide 4 (or 4), a trimer, was prepared following a modified CuAAC method by reacting 1 with a small trialkyne core, tripropargylamine, and using an excess amount Cu (I) as [(CH₃CN)₄Cu]PF₆ without sodium ascorbate. In the same manner, the trimeric peptide 5 (or 5) was prepared from 2 as a negative control for the following assays.

### Example 8

### Interaction of BK Derivatives with Lipid Vesicles

In another method, synthetic lipid vesicles of various sizes were prepared to model different membrane curvatures for testing interactions. Three compositions of vesicles were designed to closely resemble natural biological membranes named lipid model (LM) 1, 2, and 3. These vesicles were prepared by extrusion under inert, pressure-controlled method through polycarbonate membranes with pore sizes of 400, 80, and 30 nm to cover a wide range of curvatures. Since curvature is defined as the inverse of radius, the smallest size represents vesicles with the highest curvature. The described extrusion process yielded vesicles with average diameters of 464±25 nm (V₄₆₄), 116±8 nm (V₁₁₆), and 58±5 nm (V₅₈), as analyzed by dynamic light scattering (DLS). These vesicles were further characterized by transmission electron microscopy (TEM) to verify the sizes found by DLS.

Fluorescence enhancement (FE) assays were conducted to investigate if peptide 1 (see FIGS. 7A-7B) binds to lipid vesicles. Upon binding, the fluorescence intensity of a fluorophore-labeled peptide increases due to a change from aqueous polar to a hydrophobic phospholipid environment surrounding the fluorophore, concurring with blue shift of the maximum fluorescence emission wavelength. The protein C2AB from rat Synaptotagmin-1 (G374, residues 96-421), an established lipid vesicle curvature sensor, was used as positive control. The peptides (125 μL, 1 μM) were treated with lipid vesicles (125 4, 1 mM), incubated for 1 h at 4° C., and their fluorescence emission spectra recorded (λₑₓ=480 nm).

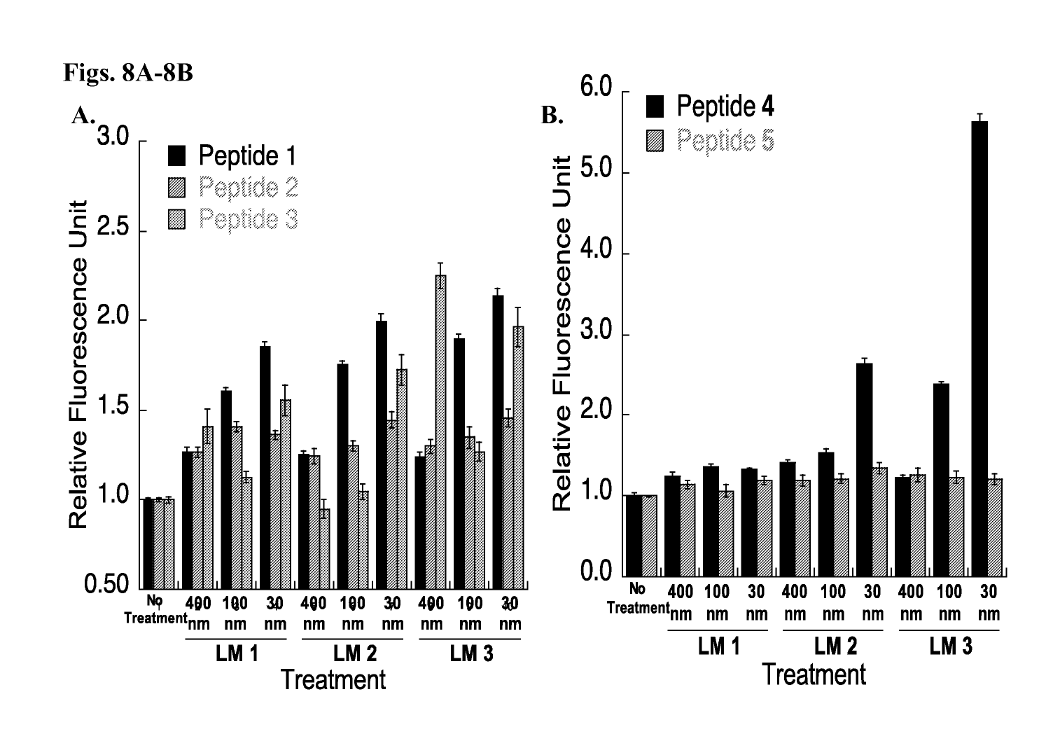

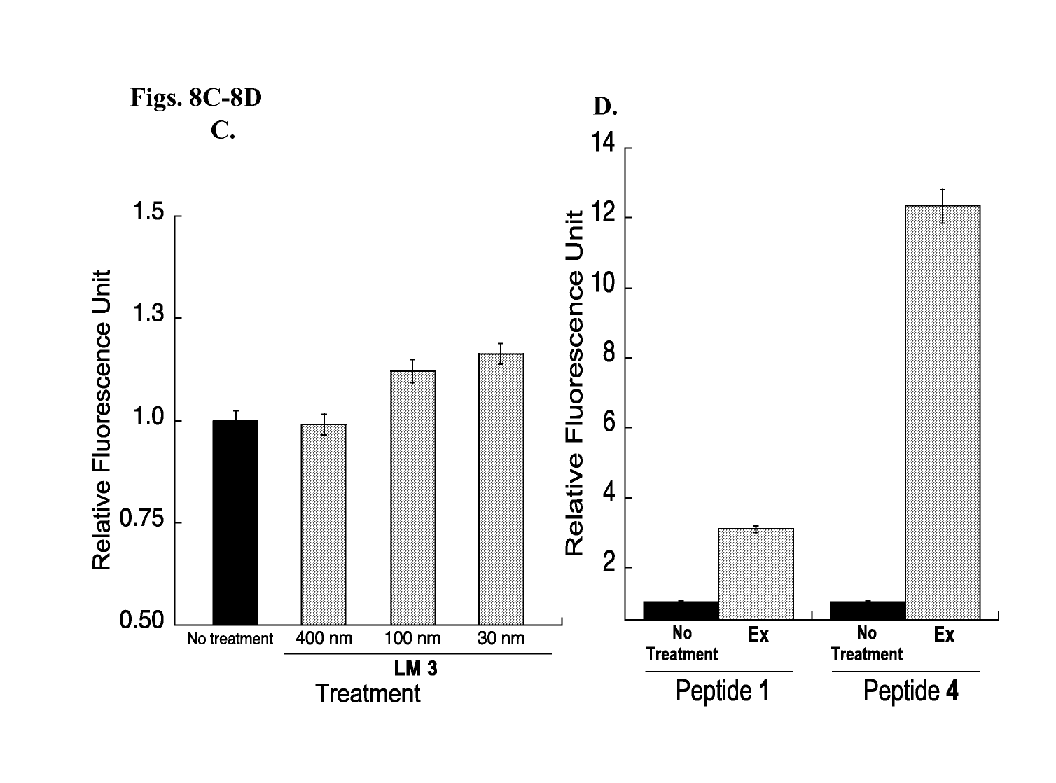

FIGS. 8A-8B are histogram plots of FE assays of BK derived peptides. FIG. 8A is a histogram plot of an FE assay of derived monomeric peptides with various lipid vesicles. Lipid vesicles V₅₈, V₁₁₆, and V₄₅₄ were prepared from LM1, LM2, and LM3 and probed with monomeric peptides 1-3. Peptide 1 treated with V₅₈ showed fluorescence intensity higher than the peptide treated with V₁₁₆, or V₄₆₄ across all three lipid models, at about two-fold increase (1.85±0.03-2.14±0.04 RFU) relative to the untreated peptide, with highest intensity observed with LM3. FIG. 8B is a histogram plot of FE assays of trimeric peptides with various vesicles. The vesicles were the same as FIG. 8A. Peptide 4 that was treated with LM3 V₅₈ showed an almost six-fold fluorescence intensity when compared to the untreated peptide (5.6±0.11 RFU), which is equivalent to a 2.6 times from that observed with 1 (FIG. 8B). Trimerization of peptide 1 into peptide 4 resulted in a significant increase in preference for lipid vesicles with d<100 and high content of the anionic lipid phosphatidylserine. These findings demonstrate that peptide 4 simultaneously recognizes membrane curvature and lipid composition, shedding insight to the mechanism of protein- or peptide-lipid interactions in membrane curvature sensing. Treatment with LM3 V₁₁₆ and LM2 V₅₈ treatment did not show a large increase in fluorescence intensity at 2.38±0.03 and 2.64±0.07 RFU, respectively. FIG. 8C provides plots of C2AB with lipid vesicles. C2AB served as a positive control. The lipid vesicles were prepared from LM3. The fluorescence intensities with V₅₈, and V₁₁₆ are significantly higher than the positive control C2AB (FIG. 8C). Meanwhile, peptide 2 demonstrated minimal fluorescence increase (1.24±0.04-1.46±0.05 RFU) upon vesicle treatment but did not show the ability of curvature differentiation. Peptide 3 strongly interacted with lipid vesicles, the highest observed with those containing higher concentration of POPS, which could be associated with non-specific electrostatic attraction. It also preferred the LM2 V₅₈, but lacks a clearly defined preference for high curvature across the different models. The circular dichroism of untreated and vesicle-treated peptides 1 and 2 was recorded following a previously reported method to probe if the presence of a membrane mimetic environment would perturb their conformations. 25% decrease in mean residue ellipticiy at 202 nm for peptide 1 treated with LM3 was found, but not for peptide 2 (data not shown). This suggests a lipid-induced conformational change of peptide 1 in the presence of LM3, consistent with BK treated with anionic micelles. The observations from FE assays were taken to imply that the lipid vesicle curvature recognition by peptide 1 and peptide 4 is affected by a combination of hydrophobic interaction due to high lipid packing defects found in V₅₈, electrostatic attraction between the peptides and the phospholipid head groups, and the claw-like orientation of the Arg residues upon peptide interaction with the lipids, the last two properties being consistent with those of BK.

In support of the observations with synthetic vesicles to translate to naturally-occurring extracellular vesicles, exosomes (Ex) purified from blood plasma of rats underwent inescapable tailshock stress as an acceptable model of exosome release. Ex have diameters of 40-100 nm, therefore providing a good biological model for testing the curvature-sensing peptides. FIG. 8D presents a histogram demonstrating an exemplary FA assay of BK derived peptides and exosomes. The total number of Ex in the sample was counted by nanoparticle tracking analysis to ensure that is in the same order of magnitude as the 500 μM lipids in the synthetic models. As illustrated in the histogram plot of FIG. 8D, the Ex treated peptides 1 and 4 have significantly higher fluorescence intensity at 3.09±0.08 and 12.34±0.47 RFU, respectively, than the untreated peptides, which demonstrated that the synthetic phospholipid vesicle sensing property of these peptides is translated to the detection of Ex. Since exosomes are known to contain ˜16-19% of the anionic phospholipids phosphatidylserine and phosphatidylinositol, amounts that correspond to those in LM3, these FE data directly correlate to findings with synthetic lipid vesicles.

In other exemplary methods, binding strength of the interactions were assessed. Fluorescence anisotropy (FA) assay was adopted to measure the binding constants. The peptide partitions between the hydrophobic lipid bilayer and the aqueous solvent and the dissociation constant (Kd) calculated as the reciprocal of the molar partition coefficient. A constant concentration of peptide was titrated with increasing concentration of lipid vesicles and the FA due to the change in the rotational time of the small peptide when it binds to the relatively bigger lipid vesicles was measured and plotted versus the total lipid concentration. Table 2 represents dissociation constant (Kd) values of various peptides in μM with different vesicles. Fluorescence anisotropy titration was performed to obtain the Kd values. Lower values indicate stronger binding. LM=liposome model. [Peptide]=1 μM; [Lipid titrant]=2 mM. As shown in Table 2, peptide 1 showed modest binding to LM2 and LM3 V₅₈ (Kd=488±72 and 407±56 μM, respectively) and modest to negligible binding for V₁₁₆ and V₄₅₄ across all lipid models. Peptide 2 showed negligible binding and peptide 3 showed modest to negligible binding to all sizes across all lipid models. Meanwhile, peptide 4 showed a Kd of 78 7 μM for LM3 V₅₈ equivalent to 6.3 fold higher that LM 3 V₁₁₆, the strongest binding strength observed among the tested peptides, and modest to weak binding for other lipid vesicles. As a comparison, C2A-C2B was reported with a Kd=151±6 μM for 105 nm vesicles. These data support that BK derivatives sense membrane curvatures and multivalency results electrostatic attraction playing a dominant role in binding strength.

TABLE 2
Dissociation constant (Kd) values of various peptides with lipid vesicles

| | Liposome Treatment | | | | | | | | |
|---|---|---|---|---|---|---|---|---|---|
| | LM 1 | | | LM 2 | | | LM 3 | | |
| Peptide | V₄₅₄ | V₁₁₆ | V₅₈ | V₄₅₄ | V₁₁₆ | V | V₄₅₄ | V₁₁₆ | V₅₈ |
| 1 | 7.14 ± 70 | 940 ± 160 | 996 ± 230 | >1000 | 585 ± 62 | 488 ± 72 | 902 ± 141 | 893 ± 101 | 407 ± 56 |
| 2 | >1000 | >1000 | >1000 | >1000 | >1000 | >1000 | >1000 | >1000 | >1000 |
| 3 | 978 ± 139 | >1000 | >1000 | 977 ± 134 | 859 ± 131 | 507 ± 70 | 706 ± 112 | 595 ± 73 | 489 ± 45 |
| 4 | 900 ± 83 | 793 ± 82 | 983 ± 247 | 790 ± 73 | 773 ± 57 | 330 ± 39 | >1000 | 489 ± 63 | 78 ± 7 |

Reported herein is the design and synthesis of an orthogonally functionalized BK derivative 1 that senses lipid vesicle curvature, independent of lipid composition. Its trimeric form, peptide 4, demonstrated higher selection and stronger affinity for lipid vesicles that are enriched in anionic phospholipids. Based on fluorescence experiments, the curvature sensing ability of these peptides could be due to electrostatic and hydrophobic attraction between the peptides and the lipid vesicles, aided by the claw-like peptide conformation that favorably orients the cationic Arg residues towards the phospholipids head groups. The synthetic lipid vesicle binding ability of these peptides directly correlated to exosomes detection. This discovery opens a new model for small peptide-based molecular probe that holds promise for curvature sensing in biological systems.

### Example 9

In one exemplary method, compositions disclosed herein can be used to assess the presence of metastatic cancer cells in a subject having cancer. In accordance with this method a health practitioner can obtain a biological sample from a subject and administer the composition to the subject prior to obtaining the biological sample, or administering the composition to the obtained biological sample without administering the composition to the subject. Then the health practitioner can assay the subject's sample for the presence or amount of lipid vesicles comprising the administered compound in the sample versus a control sample from a subject not having cancer; and detect the presence or level of lipid vesicles in the subject where the presence or amount of the lipid vesicles in the subject's sample is increased versus control thereby diagnosing the presence or level of metastatic cancer cells in the subject.

In one example, the composition can contain one or more peptide where the peptide is derived from loop 3 of the C2B domain of Synaptotagmin I represented by GGDYDKIGKNDA (SEQ ID NO:1) or GGXDYDKIGKNDANX (SEQ ID NO:13), wherein X is any cyclic linker known to one skilled in the art, for example, X at position 3 of the sequence can be L-propargylglycine, and X at position 14 can be ε-azido norleucine; b) a peptide derived a myristoylated alanine-rich C kinase substrate (MARCKS) effector domain; c) a trimeric peptide comprising peptides derived from Bradykinin; or a combination and a pharmaceutically acceptable excipient or an appropriate sample media (e.g., HEPES, saline or other physiologically appropriate media known in the art). A cancer patient contemplated herein can have liver, pancreas, cervical, kidney, lung, stomach, colon, breast, prostate, bone, skin or brain cancer.

The health practitioner may further analyze a subject's sample for lipid vesicles having a diameter of 30 nm to 100 nm targeted by the administered composition in the sample or in the subject and determine metastasis in the subject.

### Example 10

In another exemplary method, a subject undergoing surgical removal of a tumor may be further analyzed for residual cancer cells to determine whether the tumor has been completely removed. Compositions contemplated herein can be administered to the location of a tumor in the subject and presence of interaction with lipid vesicles of a predetermined size (e.g., 10 to 150 nm) can be analyzed and residual tumor cells can be identified and removed from the subject.

All of the COMPOSITIONS and/or METHODS disclosed and claimed herein can be made and executed without undue experimentation in light of the present disclosure. While the compositions and methods of this invention have been described in terms of preferred embodiments, it will be apparent to those of skill in the art that variation may be applied to the COMPOSITIONS and/or METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising:
a) a peptide derived from loop 3 of the C2B domain of Synaptotagmin I represented by GGDYDKIGKNDA (SEQ. ID NO:1) or GGXDYDKIGKNDANX (SEQ ID NO:13);
b) a peptide derived a myristoylated alanine-rich C kinase substrate (MARCKS) effector domain;
c) a trimeric peptide comprising peptides derived from Bradykinin; or
d) a combination thereof; and
a media.

2. The composition of claim 1, further comprising a side chain molecule or a tracking agent.

3. The composition of claim 2, wherein the side chain molecule or tracking agent comprises H, acetyl group, tryptophan, 4-chloro-7-nitrobenzo-2-oxa-1,3-diazole (NBD), sulfonated coumarin, sulfonated rhodamine, sulfonated xanthenes, sulfonated cyanine or other fluorophor molecule.

4. The composition of claim 1, wherein the peptide comprises 5 or more consecutive amino acids of GGDYDKIGKNDA (SEQ. ID NO:1).

5. The composition of claim 1, wherein the peptide is a) and is a cyclic peptide.

6. The composition of claim 1, wherein the peptide is a) and has a structure depicted by formula: 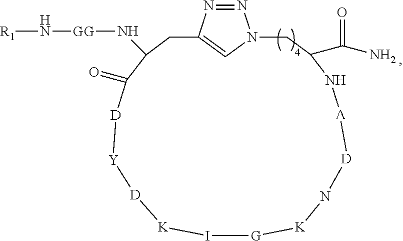
wherein R1 comprises a side chain molecule.

7. The composition of claim 1, where the cyclic peptide comprises D¹ Y² D³ K⁴ I⁵ G⁶ K⁷ N⁸ D⁹A¹⁰ having a conservative amino acid substitution or deletion at any of amino acid D¹, Y², D³, K⁴, I⁵, G⁶, K⁷, N⁸, D⁹ or A¹⁰.

8. The composition of claim 1, comprising a) wherein X is selected from a modified glycine or leucine residue or combination of both modified residues in a single peptide capable of creating a cyclic peptide.

9. The composition of claim 2, wherein the side chain is a fluorophor conjugate.

10. The composition of claim 1, comprising b) and further comprising a full-length MARCKS protein.

11. The composition of claim 1, wherein the peptide comprises b) and further comprises 5 or more, 10 or more, or 15 or more consecutive amino acids of KKKKKRFSFKKSFKLSGFSFKKNKK (SEQ ID NO: 2).

12. The composition of claim 1 comprising c) wherein the peptide further comprises a trimeric peptide of 5 or more consecutive amino acids of GRPPGFSPFR (SEQ ID NO: 3).

13. The composition of claim 1, wherein the peptide comprises a formula, 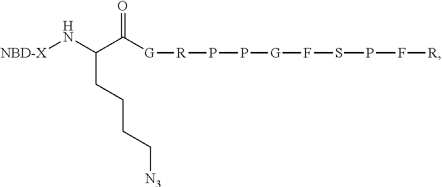
wherein NBD-X is 6-N-(7-nitrobenz-2-oxa-1,3-diazol-4-yl) aminohexanoic acid.

14. A method for detecting the presence of metastatic cancer cells in a subject having cancer, comprising:
a) obtaining a biological sample from the subject;
b) administering a pharmaceutically acceptable composition of claim 1 to the subject prior to obtaining the biological sample, or administering the composition of claim 1 to the obtained biological sample without administering a pharmaceutically acceptable composition to the subject;
c) assaying the subject's sample for presence or level of lipid vesicles comprising the administered composition in the sample versus a control without the composition; and
d) detecting the presence or level of lipid vesicles in the subject where the presence or level of the lipid vesicles in the subject's sample is increased versus control thereby diagnosing presence or level of metastatic cancer cells in the subject.

15. The method of claim 14, wherein the cancer comprises liver, pancreas, cervical, kidney, lung, stomach, colon, breast, prostate, bone, skin or brain cancer.

16. The method of claim 1, wherein c) corresponds to detecting lipid vesicles having a diameter of 10 to 150 nm in the sample or in the subject.

17. The method of claim 14, wherein c) corresponds to detecting lipid vesicles having a diameter of 30 nm to 100 nm in the sample or in the subject.

18. A method for determining location of a cancer cell in a subject comprising:
a) administering a pharmaceutically acceptable composition of claim 1 to the subject;
b) detecting location of the administered composition in the subject; and
c) identifying the location of the cancer cell in the subject according to the location of the composition.

19. A kit for identifying metastatic cell in a subject comprising:
the composition of claim 1 as a pharmaceutically acceptable formulation and a suitable container.

20. The kit of claim 19, further comprising instructions for determining the presence or location of metastatic cancer cells in a sample or in a subject.

21. The kit of claim 19, where the suitable container comprises a single dosage form device.

22. The kit of claim 19, further comprising at least a second pharmaceutically acceptable composition of claim 1.

23. The composition of claim 1, further comprising a pharmaceutically acceptable carrier.
